(19) United States
(12) Patent Application Publication
(10) Pub. No.: US 2024/0425879 A1
(43) Pub. Date: Dec. 26, 2024

(54) COMPOSITIONS AND METHODS FOR ADENO-ASSOCIATED (AAV) VIRUS DNASE EXPRESSION

(71) Applicant: CLS THERAPEUTICS, LLC, New York, NY (US)

(72) Inventors: Dmitry Genkin, Milan (IT); George Tetz, New York, NY (US)

(73) Assignee: CLS THERAPEUTICS, LLC, New York, NY (US)

(21) Appl. No.: 18/671,704

(22) Filed: May 22, 2024

Related U.S. Application Data
(60) Provisional application No. 63/468,240, filed on May 22, 2023.

(51) Int. Cl. C12N 15/86; A61K 48/00; C12N 9/22

(52) U.S. Cl. CPC C12N 15/86; A61K 48/0058; C12N 9/22; C12N 2750/14143; C12N 2830/008

(57) ABSTRACT

The invention relates to AAV gene therapy vectors containing novel deoxyribonuclease (DNase) transgene for delivery and expression of an enzyme which has a deoxyribonuclease (DNase) activity for enhanced clearance of cell-free DNA (cfDNA) accumulated intravasculary and/or extravasculary for treatment of various diseases and conditions, including cancer and neurodegeneration.

### RELATED APPLICATIONS

This is application claims the benefit of U.S. provisional application having Ser. No. 63/468,240 and a filing date of May 22, 2023.

### FIELD OF THE INVENTION

The invention relates to therapeutics, and more specifically, to AAV gene therapy vectors containing a novel deoxyribonuclease (DNase) transgene and methods of treating ailments such as cancer and neurodegeneration.

### BACKGROUND

Viral vectors are tools commonly used by molecular biologists to deliver genetic material into cells. This process can be performed inside a living organism (in vivo) or in a cell culture (in vitro). Viruses have evolved specialized molecular mechanisms to efficiently transport their genomes inside the cells they infect. Delivery of genes, or other genetic material, by a vector is termed “transduction” and the infected cells are described as “transduced.”

Major advances in gene therapy have been achieved by using viruses to deliver therapeutic genetic material. Adeno-Associated Virus (AAV) is a small, naturally occurring, non-pathogenic virus belonging to the Dependovirus genus of the Parvoviridae. Despite not causing disease, AAV is known to be able to infect humans and other primates and is prevalent in human populations. AAVs can infect a broad range of different cell types (e.g., cells of the central nervous system, heart, kidney, liver, lung, pancreas, retinal pigment epithelium or photoreceptor cells, or skeletal muscle cells). Twelve serotypes of the virus (e.g., AAV2, AAV5, AAV6, etc.) exhibiting different tissue infection capabilities (“tropisms”) have been identified. Because of its low immunogenicity and ability to effectively transduce non-dividing cells, AAV has attracted attention as a highly effective viral vector for gene therapy.

AAV is a single-stranded DNA virus that is composed of approximately 4,800 nucleotides. The viral genome may be described as having a 5′ half and a 3′ half which together comprise the genes that encode the virus' proteins. The 5′ half of the AAV genome comprises the AAV rep gene, which, through the use of multiple reading frames, staggered initiating promoters (P5, P19, and P40) and alternate splicing, encodes four non-structural Rep proteins (Rep40, Rep52, Rep68 and Rep78) that are required for viral transcription control and replication and for the packaging of viral genomes into the viral capsule. In the presence of viral proteins (such as Ad proteins), the P5 promoter becomes activated and mediates the transcription of Rep68 and Rep78 proteins, which are involved in transcriptional control, in latency, in rescue, and in viral DNA replication and thus function as master controllers of the AAV life cycle.

Expression of the Rep68 and Rep78 proteins activates the P19 promoter, which is responsible for the transcription of the Rep40 and Rep52 proteins. The 3′ half of the AAV genome comprises the AAV capsid gene (cap), which encodes three capsid proteins (VP): VP1, VP2 and VP3. The three capsid proteins are translated from a single mRNA transcript that is controlled by a single promoter (P40 in case of AAV2). The 3′ half of the AAV genome also comprises the AAP gene, which encodes the AAV assembly-activating protein (AAP). Sixty VP monomers (comprising approximately 5 copies of VP1, 5 copies of VP2, and 50 copies of VP3) self-assemble around the AAV genome to form the icosahedral protein shell (capsid) of the mature viral particle. The AAV AAP protein is believed to be required for stabilizing and transporting newly produced VP proteins from cytoplasm into the cell nucleus. The 3′ half of the AAV genome also comprises the AAV X gene, which is believed to encode a protein that supports genome replication.

AAV is an inherently defective virus, lacking the capacity to perform at least two critical functions: the ability to initiate the synthesis of viral-specific products and the ability to assemble such products to form the icosahedral protein shell (capsid) of the mature infectious viral particle. It thus requires co-infecting “helper” virus, such as adenovirus (Ad), herpes simplex virus (HSV), cytomegalovirus (CMV), vaccinia virus or human papillomavirus to provide the viral-associated (VA) RNA that is not encoded by genes of the AAV genome. Such VA RNA is not translated but plays a role in regulating the translation of other viral genes. Similarly, the AAV genome does not include genes that encode the viral proteins, E1a, E1b, E2a, and E4; thus, these proteins must also be provided by a co-infecting “helper” virus. The E1a protein greatly stimulates viral gene transcription during the productive infection. The E1b protein blocks apoptosis in adenovirus-infected cells, and thus allows productive infection to proceed. The E2a protein plays a role in the elongation phase of viral strand displacement replication by unwinding the template and enhancing the initiation of transcription. The E4 protein has been shown to affect transgene persistence, vector toxicity and immunogenicity.

AAV viruses infect both dividing and non-dividing cells and persist as circular episomal molecules or can be integrated into the DNA of a host cell at specific chromosomic loci (Adeno-Associated Virus Integration Sites or AAVS). AAV remains latent in such infected cells unless a helper virus is present to provide the functions needed for AAV replication and maturation.

In light of AAV properties, recombinantly modified versions of AAV (rAAV) have found substantial utility as vectors for gene therapy. rAAV are typically produced using circular plasmids (“rAAV plasmid vector”). The AAV rep and cap genes are typically deleted from such constructs and replaced with a promoter, a β-globin intron, a cloning site into which a therapeutic gene of choice (transgene) has been inserted, and a poly-adenylation (“polyA”) site. The inverted terminal repeated sequences (ITR) of the rAAV are, however, retained, so that the transgene expression cassette of the rAAV plasmid vector is flanked by AAV ITR sequences. Thus, in the 5′ to 3′ direction, the rAAV comprises a 5′ ITR, the transgene expression cassette of the rAAV, and a 3′ ITR.

rAAV have been used to deliver a transgene to patients suffering from any of a multitude of genetic diseases (e.g., hereditary lipoprotein lipase deficiency (LPLD), Leber's congenital amaurosis (LCA), aromatic L-amino acid decarboxylase deficiency (AADC), choroideremia and hemophilia), and have utility in new clinical modalities, such as Crispr/Cas9. More than 150 clinical trials involving rAAV have been instituted. The most commonly used AAV are transduced into cells of the central nervous system, kidney, retinal pigment epithelium and photoreceptor cells. An AAV serotype, AAV9, which infects muscle cells, has also been widely used.

Neutrophil extracellular traps (NETs) were discovered as extracellular strands of decondensed DNA, which were expelled from activated neutrophils. NETs have been implicated as key players into the pathogenesis of an increasingly large number of human diseases including cancer, acute organ injury, kidney disease, GVH disease, stroke, thrombosis, diabetes, atherosclerosis, sepsis, eclampsia, infertility, coagulopathies and neurodegeneration. Endogenous deoxyribonuclease I (DNase I) and deoxyribonuclease 1L3 (DNase IL3) enzymes activity is heavily suppressed in diseases accompanied by intensive NETs formation. It was discovered that DNase I and DNase IL3 can effectively degrade established NETs, thereby abolishing their pathogenic effects. Deoxyribonuclease enzyme is thus a useful therapeutic compound to treat pathologic conditions related to increased amount of circulating cell-free DNA and neutrophil extracellular traps.

The poor pharmacokinetic properties of natural deoxyribonuclease enzymes limit their therapeutic efficacy due to an inability to maintain meaningful DNA hydrolytic activity in blood. Industrial applicability of natural deoxyribonuclease enzymes is also limited because the quantities of enzyme required to maintain meaningful DNA hydrolytic activity in blood makes such treatment non-compliant for the patient and economically unfeasible. To overcome this unmet need in the art, it was proposed to use recombinant adeno-associated virus (rAAV) expression vectors comprising a nucleotide sequence encoding an enzyme which has a deoxyribonuclease (DNase) activity for treatment of various diseases and conditions accompanied with intravascular and extravascular accumulation of cell free DNA (cfDNA). Such use has been described in US 2019-0241908-A1, U.S. Pat. No. 11,046,943 and WO 2017/019876.

While there has been substantial progress in the use of adeno-associated virus (AAV) gene therapy vectors in real world clinical settings, setbacks related to vector toxicity and immunogenicity still represent major challenges. In many cases, these two issues appear to be inextricably linked. Immunogenicity of AAV vectors is thought to cause or exacerbate some of the more serious adverse events associated with AAV gene therapy, such as hepatotoxicity and thrombotic microangiopathy (TMA). Moreover, these adverse events tend to be correlated with vector dosage, increasing in both prevalence and severity with higher doses. Not surprisingly, high vector doses are also associated with increased immunogenicity, leading to a vicious cycle when vector doses of 1×10¹⁴ gc/kg or higher are required for efficacy (see, e.g., Takashi et al., Expert Opinion on Biological Therapy, 22:9, 1067-1071, 2022). An in-depth analysis of mortality related to AAV gene therapies by the FDA Cellular, Tissue, and Gene Therapies Advisory Committee concluded that morbidity and mortality of hepatotoxicity was only observed at AAV doses >1×10¹⁴ gc/kg. There are several approaches under development aiming to reduce clinical AAV/rAAV vector dose to improve the safety of AAV/rAAV gene therapies. For example: increasing the ratio of full/empty capsids, the use of capsids with increased transduction efficiency, provision of more powerful promoter and enhancer designs, engineering of more catalytically effective transgenes as exemplified by the Padua variant used in hemophilia B. However, such approaches carry inherent risks. For example, promoter and enhancer design can present risks of neoplastic transformation. Capsid mutations present risks of hepatotoxicity and immunotoxicity. Transgene mutations can neutralize antibody response. There thus exists a need for more efficient methods to engineer recombinant AAV DNase vectors to increase the safety and efficacy of treating diseases and conditions associated with increased levels of cfDNA in a subject's blood, tissues and body systems.

The present invention includes recombinant adeno-associated virus (rAAV) expression vectors that overcome the limitations of those conventionally used. The vectors can include a capsid protein and a nucleic acid with a promoter operably linked to a nucleotide sequence encoding an enzyme which has a deoxyribonuclease (DNase) activity. The enzyme can include at least two chorionic gonadotropin carboxy terminal peptides attached to the amino terminus or carboxy terminus. The rAAV expression vector can be used to treat diseases and conditions associated with increased levels of cfDNA in the blood and tissues (e.g., tumor growth and progression, autoimmune and neurodegenerative diseases, infections, etc.). The vector is effective when expressed in low doses (e.g., below 1.0×10¹⁴ GC/kg) and ultralow doses (e.g., below 1.0×10¹² GC/kg).

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims, drawings, and abstract as a whole.

The invention includes AVV and rAAV gene therapy vectors comprising deoxyribonuclease (DNase) transgene for delivery and expression of an enzyme which has a deoxyribonuclease (DNase) activity. The novel methods and approaches of the present invention provide enhanced clearance of cell-free DNA (cfDNA) accumulated within the vasculature or accumulated extra-vascularly.

Also known in the art as AAV and rAAV expression vectors, the gene therapy vectors of the present invention can include a capsid protein and a nucleic acid segment. In aspects, a promoter is operably linked to a nucleotide sequence encoding an enzyme which has a deoxyribonuclease (DNase) activity. In aspects, the enzyme contains at least two chorionic gonadotropin carboxy terminal peptides (CTP).

In aspects, the CTP (carboxy-terminal peptide) is derived from the naturally occurring 28 carboxy-terminal residues of human chorionic gonadotropin (hCG). The CTP has 28 amino acids and the capacity for glycosylation at four to six O-linked sugar chains, which are all at serine residue. CTP can act as a protectant against degradation of proteins and extend circulatory half-lives of proteins.

In one aspect, the chorionic gonadotropin carboxy terminal peptides are attached to the amino terminus of the expressed enzyme. In one aspect, the chorionic gonadotropin carboxy terminal peptides are attached to the carboxy terminus of the expressed enzyme.

The present invention also contemplates several capsid proteins to encapsulate the AAV and rAAV vector for therapeutic delivery. In one aspect of the invention, capsid protein LK03 is preferable as a non-limiting example. Other capsid proteins such as VP1, VP2, VP3, VP4, and all other capsid proteins suitable for the therapeutic purposes of this invention that are known in the art are inherently contemplated.

The present invention further contemplates the use of any adenovirus suitable as a vector for gene therapy delivery, gene therapy expression, and enhanced clearance of cell-free DNA accumulated either intravascularly or extravascularly.

The present invention also contemplates any Adenovirae and any Adenovirae family member that is a suitable vehicle for gene therapy, delivery and expression. Although capsid protein LK03 is preferred for encapsulating Adenovirae, any capsid protein known in the art suitable for purposes of this invention is inherently contemplated by this disclosure.

The present invention alleviates an unmet need in the art for delivering effective therapy at relatively low dosages. The pharmakoinetic properties of the present invention allow dosing of rAAV in the range between 1×10¹² gc/kg 1×10¹⁴ gc/kg in a human setting to achieve sustainable and pharmacologicaly sufficient DNA hydrolytic activity in blood; thereby avoiding use of high vector doses, and greatly reducing the incidence of toxic side effects and adverse events.

The present invention further contemplates a nucleic acid comprising a promoter operably linked to a nucleotide sequence encoding CTP-modified human hyperactive actin resistant deoxyribonuclease I enzyme. It can include three CTP molecules attached in tandem to the C-terminal. The amino acid sequence of the manufactured (CTP)-modified human hyperactive actin resistant deoxyribonuclease I enzyme is set forth in the sequence listings provided herein. In certain embodiments, the amino acid sequence is that set forth in SEQ ID NO: 1. However, other embodiments contemplate alternative sequences including: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and any other amino acid sequence known in the art as suitable for the therapeutic and/or prophylactic purposes of the present invention.

### BRIEF DESCRIPTION OF THE DRAWINGS

FIG. 1 depicts structure of DNase I chimeric genes.

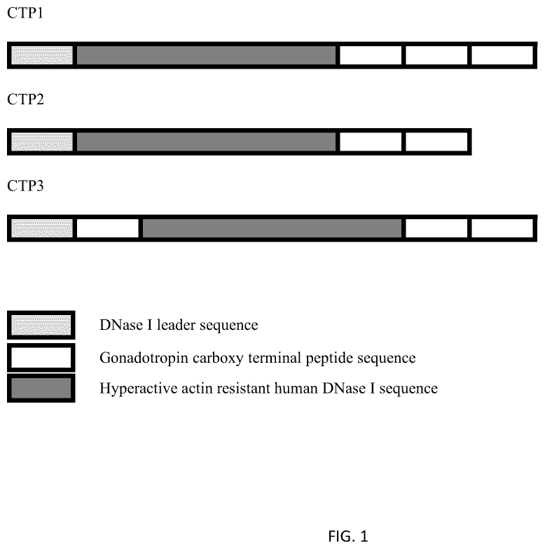

FIG. 2A is a map of the pAAV-ApoEHCR-hAATp-hDnasel-CTP1-WPRE-Xinact vector.

FIG. 2B is a map of the pAAV-Apoe HCR-hAATp-hDNaseI-CTP2-WPRE Xinact vector.

FIG. 2C is a map of the pAAV-APOE HCR-hAATp-hDnasel-CTP3-WPRE-Xinact vector.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination. Moreover, the present invention also contemplates that in some embodiments of the invention, any feature or combination of features set forth herein can be excluded or omitted.

A vector for use in gene therapy can include a virus. In an embodiment, a virus is a retrovirus, herpes simplex virus or an adenovirus.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In an embodiment, a “subject” of diagnosis or treatment is a prokaryotic or a eukaryotic cell, a tissue culture, a tissue, or an animal, e.g., a mammal, including a human.

The term “AAV” refers to adeno-associated virus and may be used to refer to the virus itself or derivatives thereof. The term covers all subtypes and both naturally occurring and recombinant forms, except where required otherwise. Adeno-associated virus (AAV), a member of the Parvovirus family, is a small nonenveloped, icosahedral virus with single-stranded linear DNA genomes of 4.7 kilobases (kb) to 6 kb. AAV is assigned to the genus, Dependovirus, because the virus was discovered as a contaminant in purified adenovirus stocks. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer. There are twelve AAV serotypes, with AAV1, AAV2, AAV4, AAV5 and AAV8. There are also different variants of AAVs, including chimerics or psuedotypes, haploids, polyploids and self-complimentary.

The term “cell free DNA” or “cfDNA” refers to free DNA molecules (e.g., of 25 nucleotides or longer) that are not contained within any intact cells. cfDNA can be measured in human blood (e.g., human serum or plasma). cfDNA is believed to be released during normal cell functions, such as secretion and export in exosomes, as well as during cell death programs, such as apoptosis and necrosis.

The term “neutrophil extracellular traps” or “NETs” refers to net-like structures composed of DNA-histone complexes and proteins released by activated neutrophils. In addition to their key role in the neutrophil innate immune response, NETs are also involved in autoimmune diseases, like systemic lupus erythematosus, rheumatoid arthritis, psoriasis, and in other non-infectious pathological processes, as coagulation disorders, thrombosis, diabetes, atherosclerosis, vasculitis, and cancer. Recently, a large body of evidence indicates that NETs are involved in cancer progression and metastatic dissemination, both in animal models and cancer patients. Endogenous deoxyribonuclease I (DNase I) and deoxyribonuclease 1L3 (DNase IL3) enzymes activity is heavily suppressed in diseases accompanied by intensive NETs formation. It was recently discovered that DNase I and DNase IL3 can effectively degrade established NETs, thereby abolishing their pathogenic effect.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease (i.e., arresting its development) and (c) relieving the disease, (i.e., causing regression of the disease).

In an embodiment, “an effective amount” refers to the amount of the defined component sufficient to achieve the desired therapeutic result. In an embodiment, that result can be effective cancer treatment.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

As used herein, the term “recombinant” refers to polypeptides or polynucleotides that do not exist naturally and which may be created by combining polynucleotides or polypeptides in arrangements that would not normally occur together. The term can refer to a polypeptide produced through a biological host, selected from a mammalian expression system, an insect cell expression system, a yeast expression system, and a bacterial expression system.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

The abbreviation “rAAV” refers to recombinant adeno-associated virus, also referred to as a recombinant AAV vector (or “rAAV vector”). The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8),

AAV type 9 (AAV-9), avian AAV, bovine AAV, canine AAV, equine AAV, primate AAV, non-primate AAV, and ovine AAV. “Primate AAV” refers to AAV that infects primates, “non-primate AAV” refers to AAV that infects non-primate mammals, “bovine AAV” refers to AAV that infect bovine mammals, etc.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsulated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component that is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA).

A “replication-competent” virus (e.g., a replication-competent AAV) refers to a phenotypically wild-type virus that is infectious and is also capable of being replicated in an infected cell (i.e., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention. Further, the viral capsid or genomic elements can contain other modifications, including insertions, deletions and/or substitutions.

A “chimeric” capsid protein as used herein means an AAV capsid protein that has been modified by substitutions in one or more (e.g., 2, 3, 4, 5, 6, 7, 8, 9, 10, etc) amino acid residues in the amino acid sequence of the capsid protein relative to wild type, as well as insertions and/or deletions of one or more (e.g., 2, 3, 4, 5, 6, 7, 8, 9, 10, etc) amino acid residues in the amino acid sequence relative to wild type. In some embodiments, complete or partial domains, functional regions, epitopes, etc., from one AAV serotype can replace the corresponding wild-type domain, functional region, epitope, etc. of a different AAV serotype, in any combination, to produce a chimeric capsid protein of this invention. Production of a chimeric capsid protein can be carried out according to protocols well known in the art and a large number of chimeric capsid proteins are described in the literature as well as herein that can be included in the capsid of this invention.

The term “variant” as used herein includes modifications or chemical equivalents of the amino acid and nucleotide sequences disclosed herein that perform substantially the same function as the proteins or nucleic acid molecules disclosed herein in substantially the same way. For example, variants of proteins disclosed herein include, without limitation, conservative amino acid substitutions. Variants of proteins disclosed herein also include additions and deletions to the proteins disclosed herein. In addition, variant peptides and variant nucleotide sequences include analogs and chemical derivatives thereof.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The amino acid substitutions may be conservative or non-conservative. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The most commonly occurring exchanges are Ala/Ser, Val/IIe, Asp/Glu, Thr/Ser, Ala/Gly, Ala/Thr, Ser/Asn, Ala/Val, Ser/Gly, Ala/Pro, Lys/Arg, Asp/Asn, Leu/IIe, Leu/Val, Ala/Glu and Asp/Gly, in both directions. Amino acid exchanges in proteins and peptides, which do not generally alter the activity of the proteins or peptides, are known in the art (H. Neurath, R. L. Hill, The Proteins, Academic Press, New York, 1979).

The term “derivative of a protein” refers to a protein having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “gene” refers to a polynucleotide containing at least one open reading frame that is capable of encoding a particular protein after being transcribed and translated.

A “transgene” refers to any gene that has been transferred from one organism to another.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

The term “control element” or “control sequence” refers to a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

The term “operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The term “expression vector” refers to a vector comprising a region which encodes a polypeptide of interest and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally occurring, wild-type AAV, and the encoded heterologous gene product is a gene product not normally encoded by a naturally occurring, wild-type AAV.

As used herein, the term “homologous recombination” means a type of genetic recombination in which nucleotide sequences are exchanged between two similar or identical molecules of DNA. Homologous recombination also produces new combinations of DNA sequences. These new combinations of DNA represent genetic variation. Homologous recombination is also used in horizontal gene transfer to exchange genetic material between different strains and species of viruses.

The terms “genetic alteration” and “genetic modification” refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be affected, for example, by infecting a cell with a recombinant plasmid or other polynucleotide through any process known in the art, such as electroporation, calcium phosphate precipitation, or contacting with a polynucleotide-liposome complex. Genetic alteration may also be affected, for example, by transduction or infection with a DNA or RNA virus or viral vector. Generally, the genetic element is introduced into a chromosome or mini chromosome in the cell; but any alteration that changes the phenotype and/or genotype of the cell and its progeny is included in this term.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273 (52): 35095-35101 (1998).

As used herein, the term “gene editing,” “Genome editing.” or “genome engineering” means a type of genetic engineering in which DNA is inserted, deleted or replaced in the genome of a living organism using engineered nucleases, or “molecular scissors”. These nucleases create site-specific double-strand breaks (DSBs) at desired locations in the genome.

As used herein, the term “gene delivery” refers to a process by which foreign DNA is transferred to host cells for applications of gene therapy.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure. The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, “gene replacement therapy” refers to administration to the recipient of exogenous genetic material encoding a therapeutic agent and subsequent expression of the administered genetic material in situ. This includes injection into the eye's vitreous. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid or protein components.

The term “ailment” refers to a disease, illness or medical condition. An ailment can be, associated with increased levels of circulating cfDNA. Examples of ailments include cancers, neurodegeneration, muscular dystrophy, amoyotrophic lateral sclerosis (ALS), inflammatory disorders and inflammation, autoimmune disorders, autoimmune deficiencies, diseases and disorders of the cardiovascular system, integumentary system, skeletal system, respiratory system, lymphatic system, endocrine system, digestive system, genetic disorders and genetic deficiencies.

The term “cancer” refers to human cancers and carcinomas, sarcomas, adenocarcinomas, etc., including solid tumors, kidney, breast, lung, kidney, bladder, urinary tract, urethra, penis, vulva, vagina, cervical, colon, ovarian, prostate, pancreas, stomach, brain, head and neck, skin, uterine, testicular, esophagus, and liver cancer. Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV.

The term “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into anti-sense RNA, as well as a “heterologous gene” (i.e., a gene encoding a protein which is not expressed or is expressed at biologically insignificant levels in a naturally occurring cell of the same type).

Alternatively, the amino acid can be a modified amino acid residue and/or can be an amino acid that is modified by post-translation modification (e.g., acetylation, amidation, formylation, hydroxylation, methylation, phosphorylation or sulfatation). A non-naturally occurring amino acid can be an “unnatural” amino acid, which can be used to chemically link molecules of interest to the AAV capsid protein or other type of viral vector.

As used herein, the term “homologous recombination” means a type of genetic recombination in which nucleotide sequences are exchanged between two similar or identical molecules of DNA. Homologous recombination also produces new combinations of DNA sequences that can represent a genetic variation. Homologous recombination is also used in horizontal gene transfer to exchange genetic material between different strains and species of viruses.

As used herein, the term “gene editing,” “Genome editing,” or “genome engineering” means a type of genetic engineering in which DNA is inserted, deleted or replaced in the genome of a living organism using engineered nucleases, or “molecular scissors.” These nucleases create site-specific double-strand breaks (DSBs) at desired locations in the genome.

As used herein, the term “gene delivery” means a process by which foreign DNA is transferred to host cells for applications of gene therapy. As used herein, the term “CRISPR” stands for Clustered Regularly Interspaced Short Palindromic Repeats, which are the hallmark of a bacterial defense system that forms the basis for CRISPR-Cas9 genome editing technology.

In some embodiments, the AAV particle of this invention can be synthetic viral vector designed to display a range of desirable phenotypes that are suitable for different in vitro and in vivo applications. Thus, in one embodiment, the present invention provides an AAV particle comprising an adeno-associated virus (AAV).

In the certain embodiments, the mammalian recipient has a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid (e.g., antisense RNA) and/or protein components.

### DETAILED DESCRIPTION OF THE INVENTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Applicant has demonstrated that systemic administration of DNase protein into a patient's circulation can treat ailments that are associated with increased levels of cfDNA in the blood. These ailments include, for example, cancers (e.g., carcinomas, sarcomas, lymphomas, melanoma; see, e.g., U.S. Pat. Nos. 7,612,032; 8,710,012; 9,248,166), development of somatic mosaicism (see, e.g., U.S. Pat. App. Pub. No. US20170056482), side effects associated with a chemotherapy or a radiation therapy (see, e.g., U.S. Pat. App. Pub. No. US20170100463), neurodegenerative diseases (see, e.g., Int. App. Pub. No. WO2016/190780), infections (see, e.g., U.S. Pat. Nos. 8,431,123 and 9,072,733), diabetes (see, e.g., U.S. Pat. No. 8,388,951), atherosclerosis (see, e.g., U.S. Pat. No. 8,388,951), stroke (see, e.g., U.S. Pat. No. 8,796,004), angina (see, e.g., U.S. Pat. No. 8,796,004), ischemia (see, e.g., U.S. Pat. No. 8,796,004), kidney damage (see, e.g., U.S. Pat. No. 9,770,492), delayed-type hypersensitivity reactions such as, e.g., graft-versus-host disease [GVHD]) (see, e.g., U.S. Pat. No. 8,535,663), reduction of fertility (see, e.g., U.S. Pat. No. 8,916,151), age-specific sperm motility impairment (see, e.g., U.S. Pat. No. 8,871,200), and aging (see, e.g., U.S. Pat. App. Pub. No. US20150110769).

The present invention provides vectors and methods for systemic delivery of a DNase into a patient's circulation to treat ailments associated with increased levels of cfDNA in the blood to treat such diseases or conditions. Accordingly, embodiments include recombinant adeno-associated virus (rAAV) expression vectors. In aspects, the vectors include a capsid protein and a nucleic acid segment with a promoter operably linked to a sequence encoding (CTP)-modified human hyperactive actin resistant deoxyribonuclease I enzyme which has a deoxyribonuclease (DNase) activity.

In aspects, the CTP (carboxy-terminal peptide) is derived from the naturally occurring 28 carboxy-terminal residues of human chorionic gonadotropin (hCG). This relatively conserved peptide (the sequence identity to its monkey and rat homologues is ˜79% and ˜74%, respectively) has been shown to provide hCG with the required longevity to maintain pregnancy (see, e.g., Matzuk, et al., Endocrinology 1990, 126, 376-383.). CTP has 28 amino acids and the capacity for glycosylation at four to six O-linked sugar chains, which are all at serine residue. Recent studies have demonstrated that CTP acts as a protectant against degradation of proteins and extends circulatory half-lives of proteins.

In embodiments, the vectors include a nucleotide sequence that encodes an enzyme which has a deoxyribonuclease (DNase) activity. The enzyme can contain at least two chorionic gonadotropin carboxy terminal peptides (CTP) attached to the amino terminus or carboxy terminus of the enzyme. When expressed in the liver, such deoxyribonuclease enzyme provides unexpectedly high levels of DNA hydrolytic activity in blood over the time. This activity is likely due to a markedly increased distribution phase and reduced clearance following secretion to blood circulation relative to the unmodified DNases. Such remarkable pharmacokinetic properties allow dosing of rAAV in the ranges described without adverse events associated with high vector doses.

Each CTP molecule typically carries four serine-linked oligosaccharides. The invention is further based on the unexpected finding that upon injection of an rAAV dose below 1.0×10¹⁴, the glycosylation profile of DNase expressed in the liver is significantly altered towards prevalence of sialylated O-link type glycans on CTP domains. This improves the pharmacologic properties of DNase by targeting of enzymes to DNA/histone filaments through interaction of the terminal sialic acid on CTP domains with positively charged residues at the ends of both histones H3 and H4. The compositions of the invention can be used for parenteral treatment of diseases related to NET formation and the presence of extracellular DNA.

Another embodiment is a recombinant adeno-associated virus (rAAV) expression vector that includes (i) a capsid protein and (ii) a nucleic acid with a promoter operably linked to a nucleotide sequence encoding (CTP)-modified human hyperactive actin resistant deoxyribonuclease I enzyme which has a deoxyribonuclease (DNase) activity. The hyperactive actin resistant deoxyribonuclease I can include three CTP molecules attached in tandem on its C-terminal end. In aspects, the amino acid sequence of the manufactured (CTP)-modified human hyperactive actin resistant deoxyribonuclease I enzyme is set forth in SEQ ID NO: 1.

Another embodiment is a recombinant adeno-associated virus (rAAV) expression vector that includes (i) a capsid protein and (ii) a nucleic acid comprising a promoter operably linked to a nucleotide sequence encoding (CTP)-modified human hyperactive actin resistant deoxyribonuclease I enzyme which has a deoxyribonuclease (DNase) activity. The said hyperactive actin-resistant deoxyribonuclease I can include two CTP molecules attached in tandem on its C-terminal end. In aspects, the amino acid sequence of the manufactured (CTP)-modified human hyperactive actin resistant deoxyribonuclease I enzyme is set forth in SEQ ID NO: 2.

Another embodiment is a recombinant adeno-associated virus (rAAV) expression vector. The vector can include (i) a capsid protein and (ii) a nucleic acid comprising a promoter operably linked to a nucleotide sequence encoding (CTP)-modified human hyperactive actin resistant deoxyribonuclease I enzyme which has a deoxyribonuclease (DNase) activity. The hyperactive actin resistant deoxyribonuclease I can include two CTP molecules attached in tandem on its C-terminal end and one CTP molecule attached on its N-terminal end. In aspects, the amino acid sequence of the manufactured (CTP)-modified human hyperactive actin resistant deoxyribonuclease I enzyme is set forth in SEQ ID NO: 3.

Another embodiment is a recombinant adeno-associated virus (rAAV) expression vector that includes (i) a capsid protein and (ii) a nucleic acid comprising a promoter operably linked to a nucleotide sequence encoding (CTP)-modified human hyperactive actin resistant deoxyribonuclease I enzyme which has a deoxyribonuclease (DNase) activity. The hyperactive actin resistant deoxyribonuclease I can include two CTP molecules attached in tandem on its C-terminal end and one CTP molecule attached on its N-terminal end. In aspects, the amino acid sequence of the manufactured (CTP)-modified human hyperactive actin resistant deoxyribonuclease I enzyme is set forth in one of the following amino acid sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14.

Another embodiment is a recombinant adeno-associated virus (rAAV) expression vector that includes (i) a capsid protein and (ii) a nucleic acid comprising a promoter operably linked to a nucleotide sequence encoding CTP-modified human hyperactive actin resistant deoxyribonuclease I enzyme that includes three CTP molecules attached in tandem to the C-terminal end. Upon expression of the rAAV in liver cells, the CTP modified hyperactive actin resistant deoxyribonuclease I enzyme has a high sialic acid content. The CTP-modified hyperactive actin resistant deoxyribonuclease I enzyme can have the amino acid sequence set forth in SEQ ID NO: 1.

The vectors have improved pharmacokinetic properties over conventional vectors. Specifically, dosing of rAAV can be achieved in the range between 1×10¹² gc/kg 1×10¹⁴ gc/kg in humans to achieve sustainable and pharmacologicaly sufficient DNA hydrolytic activity in blood. This avoids the necessity of high vector doses that can lead to toxicity and adverse events

Another embodiment is a method of treating a subject having disease or condition accompanied by intravascular or extravascular accumulation of extracellular DNA. The method can include administering a therapeutically effective amount of recombinant adeno-associated virus (rAAV) expression vector. The rAAV expression vector can include (i) a capsid protein and (ii) a nucleic acid with a promoter operably linked to a nucleotide sequence encoding CTP-modified human hyperactive actin resistant deoxyribonuclease I enzyme that has three CTP molecules attached in tandem to the C-terminal.

Another embodiment is a method of reducing the rAAV toxicity in a subject, having disease or condition accompanied by intravascular or extravascular accumulation of extracellular DNA. The method can include administering a dose of between 1×10¹² gc/kg and 1×10¹⁴ gc/kg of recombinant adeno-associated virus (rAAV) expression vector. The rAAV expression vector can include (i) a capsid protein and (ii) a nucleic acid comprising a promoter operably linked to a nucleotide sequence encoding CTP-modified human hyperactive actin resistant deoxyribonuclease I enzyme with three CTP molecules attached in tandem to the C-terminal.

Another embodiment is a method of treating a subject having a disease or condition accompanied by intravascular or extravascular accumulation of extracellular DNA. The method can include administering a therapeutically effective amount of recombinant adeno-associated virus (rAAV) expression vector. The rAAV expression vector can include (i) a capsid protein and (ii) a nucleic acid comprising a promoter operably linked to a nucleotide sequence encoding CTP-modified human hyperactive actin resistant deoxyribonuclease I enzyme with three CTP molecules attached in tandem to the C-terminal. Upon expression of the rAAV in liver cells, the CTP modified hyperactive actin resistant deoxyribonuclease I enzyme has a high sialic acid content. Moreover, the modified hyperactive actin resistant deoxyribonuclease with high sialic acid has increased affinity to DNA/histone filaments and forms complexes with DNA/histone filaments.

### Carboxy Terminal Peptide Variations

In one embodiment, the carboxy terminal peptide (CTP) has the amino acid sequence from amino acid 112 to position 145 of human chorionic gonadotrophin, as set forth in SEQ ID NO: 10. In another embodiment, the CTP sequence has the amino acid sequence from amino acid 118 to position 145 of human chorionic gonadotropin, as set forth in SEQ ID NO: 11. In another embodiment, the CTP sequence also commences from any position between positions 112-118 and terminates at position 145 of human chorionic gonadotrophin. In some embodiments, the CTP sequence peptide is 28, 29, 30, 31, 32, 33 or 34 amino acids long and commences at position 112, 113, 114, 115, 116, 117 or 118 of the CTP amino acid sequence.

In another embodiment, the CTP peptide is a variant of chorionic gonadotrophin CTP which differs from the native CTP by 1-5 conservative amino acid substitutions as described in U.S. Pat. No. 5,712,122. In another embodiment, the CTP peptide is a variant of chorionic gonadotrophin CTP which differs from the native CTP by 1 conservative amino acid substitution. In another embodiment, the CTP peptide is a variant of chorionic gonadotrophin CTP which differs from the native CTP by 2 conservative amino acid substitutions. In another embodiment, the CTP peptide is a variant of chorionic gonadotrophin CTP which differs from the native CTP by 3 conservative amino acid substitutions. In another embodiment, the CTP peptide is a variant of chorionic gonadotrophin CTP which differs from the native CTP by 4 conservative amino acid substitutions. In another embodiment, the CTP peptide is a variant of chorionic gonadotrophin CTP which differs from the native CTP by conservative amino acid substitutions. In another embodiment, the CTP peptide amino acid sequence of the present invention is at least 70% homologous to the native CTP amino acid sequence or a peptide thereof. In another embodiment, the CTP peptide amino acid sequence of the present invention is at least 80% homologous to the native CTP amino acid sequence or a peptide thereof. In another embodiment, the CTP peptide amino acid sequence of the present invention is at least 90% homologous to the native CTP amino acid sequence or a peptide thereof. In another embodiment, the CTP peptide amino acid sequence of the present invention is at least 95% homologous to the native CTP amino acid sequence or a peptide thereof.

In one embodiment, at least one of the chorionic gonadotrophin CTP amino acid sequences is truncated. In another embodiment, both of the chorionic gonadotrophin CTP amino acid sequences are truncated. In another embodiment, 2 of the chorionic gonadotrophin CTP amino acid sequences are truncated. In another embodiment, 2 or more of the chorionic gonadotrophin CTP amino acid sequences are truncated. In another embodiment, all of the chorionic gonadotrophin CTP amino acid sequences are truncated. In another embodiment, all but one of the chorionic gonadotrophin CTP amino acid sequences are truncated. In another embodiment, all but two of the chorionic gonadotrophin amino acid sequences are truncated. In one embodiment, the truncated CTP comprises the first 10 amino acids of SEQ ID NO: 12. In one embodiment, the truncated CTP comprises the first 11 amino acids of SEQ ID NO: 12. In one embodiment, the truncated CTP comprises the first 12 amino acids of SEQ ID NO: 12. In one embodiment, the truncated CTP comprises the first 13 amino acids of SEQ ID NO: 12. In one embodiment, the truncated CTP comprises the first 14 amino acids of SEQ ID NO: 12. In one embodiment, the truncated CTP comprises the first 15 amino acids of SEQ ID NO: 12. In one embodiment, the truncated CTP comprises the first 16 amino acids of SEQ ID NO: 12. DNase Variations

Specific non-limiting examples of enzymes which have a DNase activity that can be used in the compositions and methods of the invention include DNase I, DNase X, DNase γ, DNase1L1, DNase1L2, DNase 1L3, DNase II (e.g., DNase 11α, DNase IIβ), caspase-activated DNase (CAD), endonuclease G (ENDOG), granzyme B (GZMB), phosphodiesterase I, lactoferrin, acetylcholinesterase, and mutants or derivatives thereof. If the enzyme which has a DNase activity is DNase I, various mutants weakening actin-binding may be used. Specific non-limiting examples of residues in wild-type recombinant human DNase I (SEQ ID NO: 4) that can be mutated include: Gln-9, Glu-13, Thr-14, His-44, Asp-53, Tyr-65, Val-66, Val-67, Glu-69, Asn-74, and Ala-114. In various embodiments, the Ala-114 mutation is used. For example, in human DNase I hyperactive mutant comprising the sequence of SEQ ID NO: 5, the Ala-114 residue is mutated. Complementary residues in other DNases may also be mutated. Specific non-limiting examples of mutations in wild-type human recombinant DNAse I include H44C, H44N, L45C, V48C, G49C, L52C, D53C, D53R, D53K, D53Y, D53A, N56C, D58S, D58T, Y65A, Y65E, Y65R, Y65C, V66N, V67E, V67K, V67C, E69R, E69C, A114C, A114R, H44N: T46S, D53R: Y65A, D53R: E69R, H44A: D53R: Y65A, H44A: Y65A: E69R, H64N: V66S, H64N: V66T, Y65N: V67S, Y65N: V67T, V66N: S68T, V67N: E69S, V67N: E69T, S68N: P70S, S68N: P70T, S94N: Y96S, S94N: Y96T. Various DNase mutants for increasing DNase activity may be used. Specific non-limiting examples of mutations in wild-type human recombinant DNAse I include, e.g., Gln-9, Glu-13, Thr-14, His-44, Asp-53, Tyr-65, Val-66, Val-67, Glu-69, Asn-74, and Ala-114. Specific non-limiting examples of mutations for increasing the activity of wild-type human recombinant DNase I include Q9R, E13R, E13K, T14R, T14K, H44R, H44K, N74K, and A114F. For example, a combination of the Q9R, E13R, N74K and A114F mutations may be used, with such combination found at least in the hyperactive DNase I mutant comprising the sequence of SEQ ID NO: 5. WO2021168413 (A1) describes certain mutants and and derivatives of DNase1L2, and DNase 1L3 enzymes suitable for use in the present invention.

### AAV Capsid Variations

AAV vectors disclosed herein may be derived from any AAV serotype, including combinations of serotypes (e.g., “pseudotyped” AAV) or from various genomes (e.g., single-stranded or self-complementary). A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences). Non-limiting examples of AAV serotypes which can be used to develop the AAV expression vectors of the invention include, e.g., AAV serotype 1 (AAV1), AAV2, AAV3 (including types 3A and 3B), AAV4, AAV5, AAV6, AAV7, AAV8, AAV9, AAV10, AAV11, AAV12, AAVrh10 (as disclosed, e.g., in U.S. Pat. No. 9,790,472, International Patent Application Pub. No. WO2017/180857 and WO2017/180861), AAVLK03 (as disclosed, e.g., in Wang et al., Mol. Ther., 2015, 23 (12): 1877-1887), AAVhu37 (as disclosed, e.g., in Int. Pat. Appl. Pub. No. WO2017180857), AAVrh64R1 (as disclosed, e.g., in Int. Pat. Appl. Pub. No. WO2017180857), Anc80 (based on a predicted ancestor of serotypes AAV1, AAV2, AAV8 and AAV9; see Zinn et al., Cell Rep., 2015, 12 (67): 1056-1068), avian AAV, bovine AAV, canine AAV, equine AAV, ovine AAV, and any other AAV now known or later discovered. See, e.g., Fields et al., Virology, volume 2, chapter 69 (4th ed., Lippincott-Raven Publishers). Recently, a number of putative new AAV serotypes and clades have been identified (see, e.g., Gao et al., (2004) J. Virology 78:6381-6388; Moris et al., (2004) Virology 33-: 375-383). The genomic sequences of the various serotypes of AAV and the autonomous parvoviruses, as well as the sequences of the terminal repeats, Rep proteins, and capsid subunits are known in the art, by way of example, Srivistava et al., (1983) J. Virology 45:555; Chiorini et al., (1998) J. Virology 71:6823; Chiorini et a1, (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-: 375-383; GenBank Accession number U89790; GenBank Accession number J01901; GenBank Accession number AF043303; GenBank Accession number AF085716; GenBank Accession number NC 006152; GenBank Accession number Y18065; GenBank Accession number NC 006260; GenBank Accession number NC_006261; International Patent Publication Nos. WO 00/28061, WO 99/61601, WO 98/11244; and U.S. Pat. No. 6,156,303.

### EXAMPLES

The present invention is also described and demonstrated by way of the following examples.

### Example 1

### Cloning of AAV Vector: ApoEHCR Enhancer-hAAT Promoter-hDNaseI (Hyperactive) Leader CTP-WPRE Xinact Constructs

Gibson assembly is a molecular cloning method that allows for the joining of multiple DNA fragments in a single, isothermal reaction. A Gibson assembly strategy was used to generate a ApoEHCR enhancer-hAAT promoter-hDNaseI (hyperactive) leader CTP-WPRE Xinact constructs. First a ˜1 kb fragment from the original plasmid, pAAV-ApoEHCR enhancer-hAAT prometer-hDNaseI (hyperactive) correct leader-WPRE was cut out. This step removed the entire DNaseI fragment and some flanking regions on both sides.

Step 1: Digest pAAV-ApoEHCR enhancer-hAAT prometer-hDNaseI (hyperactive) correct leader-WPRE plasmid. The plasmid is cut with BamHI and NheI. Next, gel extract the 4698 bp fragment which is the plasmid backbone.

Step 2: Gibson ds DNA fragments are created.

hDNaseI (Hyper) CTP1 Gibson Fragment:

TGGTTAATATTCACCAGCAGCCTCCCCCGTTGCCCCTCT
GGGATCCACTGCTTAAATACGGACGAGGACAGGGCCCTG
TCTCCTCAGCTTCAGGCACCACCACTGACCTGGGACAGT
GAATGCCGCCACCATGAGGGGCATGAAGCTGCTGGGGGC
GCTGCTGGCACTGGCGGCCCTACTGCAGGGGGCCGTGTC
CCTGAAGATCGCAGCCTTCAACATCAGGACATTTGGGAG
GACCAAGATGTCCAATGCCACCCTCGTCAGCTACATTGT
GCAGATCCTGAGCCGCTATGACATCGCCCTGGTCCAGGA
GGTCAGAGACAGCCACCTGACTGCCGTGGGGAAGCTGCT
GGACAACCTCAATCAGGATGCACCAGACACCTATCACTA
CGTGGTCAGTGAGCCACTGGGACGGAAGAGCTATAAGGA
GCGCTACCTGTTCGTGTACAGGCCTGACCAGGTGTCTGC
GGTGGACAGCTACTACTACGATGATGGCTGCGAGCCCTG
CGGGAACGACACCTTCAACCGAGAGCCATTCATTGTCAG
GTTCTTCTCCCGGTTCACAGAGGTCAGGGAGTTTGCCAT
TGTTCCCCTGCATGCGGCCCCGGGGGACGCAGTAGCCGA
GATCGACGCTCTCTATGACGTCTACCTGGATGTCCAAGA
GAAATGGGGCTTGGAGGACGTCATGTTGATGGGCGACTT
CAATGCGGGCTGCAGCTATGTGAGACCCTCCCAGTGGTC
ATCCATCCGCCTGTGGACAAGCCCCACCTTCCAGTGGCT
GATCCCCGACAGCGCTGACACCACAGCTACACCCACGCA
CTGTGCCTATGACAGGATCGTGGTTGCAGGGATGCTGCT
CCGAGGCGCCGTTGTTCCCGACTCGGCTCTTCCCTTTAA
CTTCCAGGCTGCCTATGGCCTGAGTGACCAACTGGCCCA
AGCCATCAGTGACCACT
ATCCAGTGGAGGTGATGCTGAAGTCC TCC AGT AGT AAG
GCC CCA CCT CCC AGT TTG CCC AGT CCT TCC AGG
TTG CCA GGG CCA AGT GAC ACC CCT ATT TTG CCA
CAA TCA TCT AGC AGT AAA GCA CCA CCA CCT TCT
CTT CCT TCT CCC AGC AGA TTG CCC GGA CCC TCT
GAT ACT CCC ATA TTG CCC CAG TCC TCT TCT TCA
AAG GCC CCT CCT CCC TCA CTT CCA TCT CCT AGC
AGG CTT CCC GGA CCT TCA GAT ACA CCA ATC CTG
CCT CAG TGAAGTGGCGGCCGCTCGAGCTAGCCGGCCGCTC
TAGAAGATAATCAACCTCTGGATTACAAAAT

hDNaseI (Hyper) CTP2 Gibson Fragment:

TGGTTAATATTCACCAGCAGCCTCCCCCGTTGCCCCTCTG
GGATCCACTGCTTAAATACGGACGAGGACAGGGCCCTGTC
TCCTCAGCTTCAGGCACCACCACTGACCTGGGACAGTGAA
TGCCGCCACCATGAGGGGCATGAAGCTGCTGGGGGCGCTG
CTGGCACTGGCGGCCCTACTGCAGGGGGCCGTGTCCCTGA
AGATCGCAGCCTTCAACATCAGGACATTTGGGAGGACCAA
GATGTCCAATGCCACCCTCGTCAGCTACATTGTGCAGATC
CTGAGCCGCTATGACATCGCCCTGGTCCAGGAGGTCAGAG
ACAGCCACCTGACTGCCGTGGGGAAGCTGCTGGACAACCT
CAATCAGGATGCACCAGACACCTATCACTACGTGGTCAGT
GAGCCACTGGGACGGAAGAGCTATAAGGAGCGCTACCTGT
TCGTGTACAGGCCTGACCAGGTGTCTGCGGTGGACAGCTA
CTACTACGATGATGGCTGCGAGCCCTGCGGGAACGACACC
TTCAACCGAGAGCCATTCATTGTCAGGTTCTTCTCCCGGT
TCACAGAGGTCAGGGAGTTTGCCATTGTTCCCCTGCATGC
GGCCCCGGGGGACGCAGTAGCCGAGATCGACGCTCTCTAT
GACGTCTACCTGGATGTCCAAGAGAAATGGGGCTTGGAGG
ACGTCATGTTGATGGGCGACTTCAATGCGGGCTGCAGCTA
TGTGAGACCCTCCCAGTGGTCATCCATCCGCCTGTGGACA
AGCCCCACCTTCCAGTGGCTGATCCCCGACAGCGCTGACA
CCACAGCTACACCCACGCACTGTGCCTATGACAGGATCGT
GGTTGCAGGGATGCTGCTCCGAGGCGCCGTTGTTCCCGAC
TCGGCTCTTCCCTTTAACTTCCAGGCTGCCTATGGCCTGA
GTGACCAACTGGCCCAAGCCATCAGTGACCACT
ATCCAGTGGAGGTGATGCTGAAGTCC AGC AGT AGT
AAA GCT CCA CCC CCT TCC TTG CCA AGT CCC
TCT CGG CTC CCA GGT CCA AGC GAT ACC CCC
ATT CTG CCT CAA AGT AGT AGC AGC AAA GCA
CCT CCT CCT AGT CTT CCA AGT CCA AGT CGA
CTG CCA GGG CCA AGT GAC ACC CCC ATA TTG
CCC CAGTGAAGTGGCGGCCGCTCGAGCTAGCCGGCCG
CTCTAGAAGATAATCAACCTCTGGATTACAAAAT

hDNaseI (Hyper) CTP3 Gibson Fragment:

TGGTTAATATTCACCAGCAGCCTCCCCCGTTGCCCCTCTGGGATCC
ACTGCTTAAATACGGACGAGGACAGGGCCCTGTCTCCTCAGCTTCA
GGCACCACCACTGACCTGGGACAGTGAATGCCGCCACCATGAGGGG
CATGAAGCTGCTGGGGGCGCTGCTGGCACTGGCGGCCCTACTGCAG
GGGGCCGTGTCC TCA AGT TCC TCC AAG GCA CCC CCA
CCT AGT CTT CCT AGC CCC AGT CGC CTC CCC GGA
CCT TCA GAC ACT CCT ATT CTG CCA CAA
CTGAAGATCGCAGCCTTCAACATCAGGACATTTGGGAGGACCAAGA
TGTCCAATGCCACCCTCGTCAGCTACATTGTGCAGATCCTGAGCCG
CTATGACATCGCCCTGGTCCAGGAGGTCAGAGACAGCCACCTGACT
GCCGTGGGGAAGCTGCTGGACAACCTCAATCAGGATGCACCAGACA
CCTATCACTACGTGGTCAGTGAGCCACTGGGACGGAAGAGCTATAA
GGAGCGCTACCTGTTCGTGTACAGGCCTGACCAGGTGTCTGCGGTG
GACAGCTACTACTACGATGATGGCTGCGAGCCCTGCGGGAACGACA
CCTTCAACCGAGAGCCATTCATTGTCAGGTTCTTCTCCCGGTTCAC
AGAGGTCAGGGAGTTTGCCATTGTTCCCCTGCATGCGGCCCCGGGG
GACGCAGTAGCCGAGATCGACGCTCTCTATGACGTCTACCTGGATG
TCCAAGAGAAATGGGGCTTGGAGGACGTCATGTTGATGGGCGACTT
CAATGCGGGCTGCAGCTATGTGAGACCCTCCCAGTGGTCATCCATC
CGCCTGTGGACAAGCCCCACCTTCCAGTGGCTGATCCCCGACAGCG
CTGACACCACAGCTACACCCACGCACTGTGCCTATGACAGGATCGT
GGTTGCAGGGATGCTGCTCCGAGGCGCCGTTGTTCCCGACTCGGCT
CTTCCCTTTAACTTCCAGGCTGCCTATGGCCTGAGTGACCAACTGG
CCCAAGCCATCAGTGACCACTATCCAGTGGAGGTGATGCTGAAGTCA
TCT AGT TCC AAG GCT CCT CCC CCT TCC TTG CCA
TCT CCA TCC CGG TTG CCC GGT CCC TCA GAC ACC
CCT ATT CTT CCC CAA AGT TCC AGT TCT AAA GCT
CCT CCC CCC AGT CTT CCT TCC CCT AGC AGG CTC
CCC GGA CCA TCC GAC ACT CCC ATA TTG CCT
CAATGAAGTGGCGGCCGCTCGAGCTAGCCGGCCGCTCTAGAA
GATAATCAACCTCTGGATTACAAAAT

Step 3: Gibson Assembly. The Gibson assembly was the same for each of the Gibson fragments. The vector backbone plasmid is taken from Step 1. Each of the different DNaseI-CTP fragments are taken from step 2.

Three assemblies, one for each CTP were made. The final three plasmids were:
1. pAAV-ApoEHCR-hAATp-hDnasel-CTP1-WPRE-Xinact
2. pAAV-Apoe HCR-hAATp-hDNaseI-CTP2-WPRE Xinact
3. pAAV-APOEHCR-hAATp-hDnasel-CTP3-WPRE-Xinact
A. NEB Gibson Assembly Master mix E2611S was used.
B. An assembly reaction was set up and the mix was incubated at 50° C. for 15 min.
C. Transform into electrocompetent SURE cells (or other suitable bacteria strain that maintains AAV ITR integrity)

TABLE 1

| Reagent | Volume |
|---|---|
| Gibson master mix (2x) | 10 μl |
| Vector | TBD (50 ng) |
| DNAse I-CTP fragment | TBD (50 ng) |
| Water | Up to 20 μl |

### Example 2

### Method of Treating of Prostate Cancer

In this example, a patient presents signs and symptoms of prostate cancer. A health care practitioner confirms the diagnosis through a biopsy. Additional testing confirms elevated levels of cell-free DNA (cfDNA).

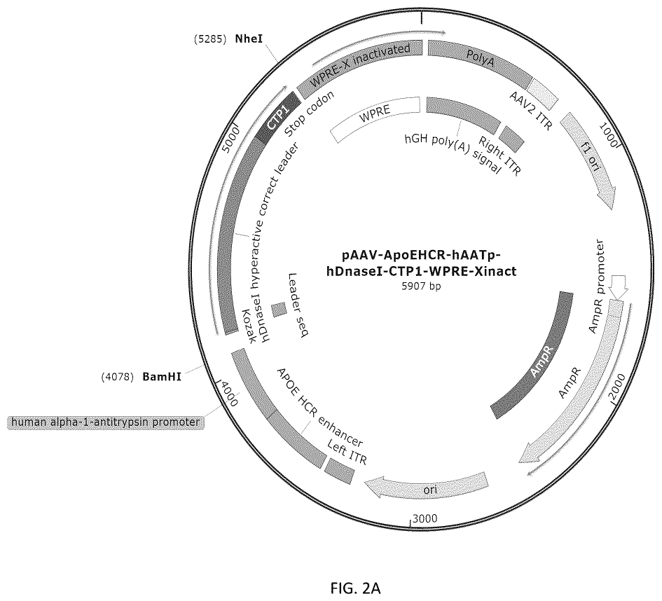

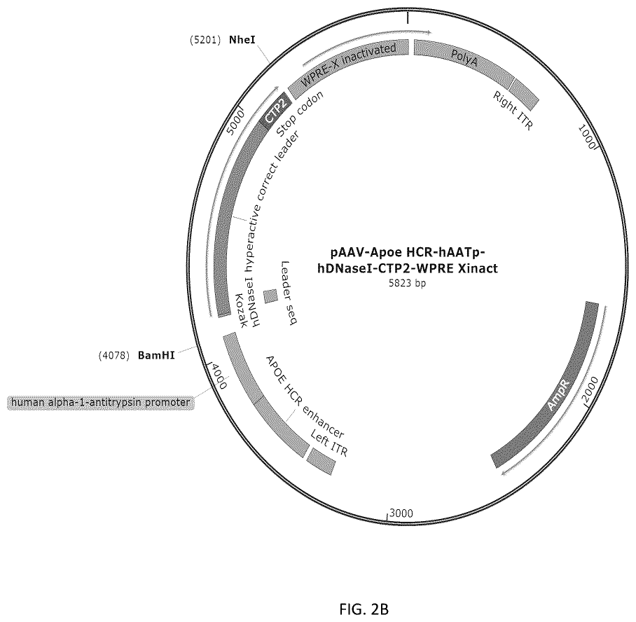

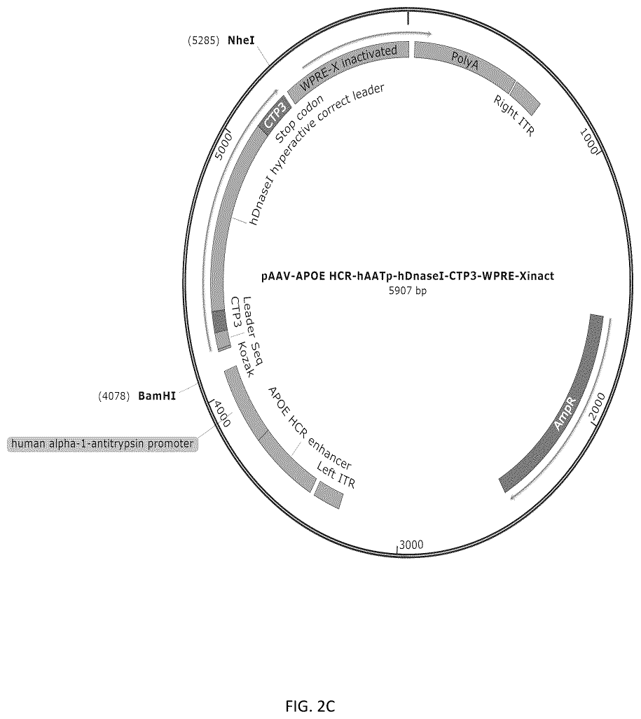

The health care practitioner devises a treatment plan that includes administering an adeno-associated virus (AAV) gene therapy vector to express an enzyme with deoxyribonuclease (DNase) activity. Specifically, the vector depicted in FIG. 2A is administered systemically to the patient at a low dose (i.e., 1.0×10¹⁴ GC/kg) to clear cfDNA.

The patient is tested periodically to determine progression of the disease. The patient can be given additional therapeutics (e.g., biologics) to help him remain in remission and/or slow progression of the cancer.

### Example 3

### Method of Treating of Breast Cancer

In this example, a patient visits a health care provider for a routine screening. The patient has tissue growth suggestive of breast cancer. The health care practitioner confirms the diagnosis through a biopsy. Additional testing confirms elevated levels of cell-free DNA (cfDNA).

The health care practitioner devises a treatment plan that includes removal of the tumor and nearby tissue, administration of a chemotherapeutic reagent (e.g., an alkylating agent) and administration of an adeno-associated virus (AAV) gene therapy vector to express an enzyme with deoxyribonuclease (DNase) activity. Specifically, the vector depicted in FIG. 2B is administered systemically to the patient at a low dose (i.e., 1.0×10¹⁴ GC/kg) to clear cfDNA. The patient is tested periodically to determine progression of the disease.

### Example 4

### Method of Treating Neurodegeneration

In this example, a patient presents signs and/or symptoms of Parkinson's disease. A health care practitioner confirms the diagnosis through an assesses of the patient's medical history and neurological examination.

The health care practitioner devises a treatment plan that includes administering an adeno-associated virus (AAV) gene therapy vector to express an enzyme with deoxyribonuclease (DNase) activity. Specifically, the vector depicted in FIG. 2B is administered systemically to the patient at a low dose (i.e., 1.0×10¹⁴ GC/kg) to clear cfDNA.

The patient is tested periodically to determine progression of the disease. Imaging (e.g., DaTscan) can also be conducted. The patient can be administered other medicaments and undergo physical therapy to help slow progression of the disease.

### Methods of Use

Embodiments include a method of treating a disease or condition and/or slowing the aging process or reducing signs of aging. The method can include administering an agent that includes administering an adeno-associated virus (rAAV) to express a DNAse. In one embodiment, a method includes administering to a pharmaceutical formulation containing a therapeutic agent that reduces levels of cell free DNA (cfDNA). A treatment regimen can include administering a pharmaceutical formulation for a time sufficient and in an amount sufficient to achieve a clinically significant reduction.

The therapeutic method of the present specification can include the step of administering drug product at a pharmaceutically effective amount. The total daily dose should be determined through appropriate medical judgment by a physician and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In one embodiment, a therapeutic disclosed herein is capable of reducing signs/symptoms in an individual suffering from a disease or condition as described herein by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms in an individual suffering from a disease or condition by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In aspects, a pharmaceutical composition compound disclosed herein reduces the size of a tumor by, e.g., at least 10%, at least 15%, at least 20%, at least 25%, at least 30%, at least 35%, at least 40%, at least 45%, at least 50%, at least
55%, at least 60%, at least 65%, at least 70%, at least 75%, at least 80%, at least 85%, at least 90% or at least 95%. In yet other aspects of this embodiment, a pharmaceutical composition disclosed herein reduces the size of a tumor from, e.g., about 5% to about 100%, about 10% to about 100%, about 20% to about 100%,
about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In a further embodiment, a therapeutic and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a therapeutic disclosed herein reduces signs/symptoms in an individual suffering from a disease or condition by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces signs/symptoms by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces signs/symptoms by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutically effective amount of a therapeutic disclosed herein reduces the aging process and/or reduces signs of aging in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces the aging process and/or reduces signs of aging by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces the aging process and/or reduces signs of aging by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter. Further, the use of the terms “include,” “includes” and “including” means include, includes and or including as well as include, includes and including, but not limited to.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

TABLE 2
SEQUENCES

| SEQ ID | SEQUENCE | |
|---|---|---|
| NO. | DESCRIPTION | SEQUENCE |
| SEQ ID | Amino acid sequence of | LKIAAFNIRTFGRTKMSNATLVSYIVQILSRYDIALVQ |
| NO: 1 | hyperactive actin resistant | EVRDSHLTAVGKLLDNLNQDAPDTYHYVVSEPLGRKSY |
| | human Dnasel compising | KERYLFVYRPDQVSAVDSYYYDDGCEPCGNDTFNREPF |
| | three chorionic | IVRFFSRFTEVREFAIVPLHAAPGDAVAEIDALYDVYL |
| | gonadotropin carboxy | DVQEKWGLEDVMLMGDFNAGCSYVRPSQWSSIRLWTS |
| | terminal peptides (CTP) | PTFQWLIPDSADTTATPTHCAYDRIVVAGMLLRGAVV |
| | attached to the carboxy | PDSALPFNFQAAYGLSDQLAQAISDHYPVEVMLKSSS |
| | terminus (CTP1) | SKAPPPSLPSPSRLPGPSDTPILPQSSSSKAPPPSLP |
| | | SPSRLPGPSDTPILPQSSSSKAPPPSLPSPSRLPGPS |
| | | DTPILPQ |
| SEQ ID | Amino acid sequence of | LKIAAFNIRTFGRTKMSNATLVSYIVQILSRYDIALVQ |
| NO: 2 | hyperactive actin resistant | EVRDSHLTAVGKLLDNLNQDAPDTYHYVVSEPLGRKSY |
| | human Dnasel compising | KERYLFVYRPDQVSAVDSYYYDDGCEPCGNDTFNREPF |
| | two chorionic gonadotropin | IVRFFSRFTEVREFAIVPLHAAPGDAVAEIDALYDVYL |
| | carboxy terminal peptides | DVQEKWGLEDVMLMGDFNAGCSYVRPSQWSSIRLWTSP |
| | (CTP)-attached to the | TFQWLIPDSADTTATPTHCAYDRIVVAGMLLRGAVVPD |
| | carboxy terminus (CTP2) | SALPFNFQAAYGLSDQLAQAISDHYPVEVMLKSSSSKA |
| | | PPPSLPSPSRLPGPSDTPILPQSSSSKAPPPSLPSPSR |
| | | LPGPSDTPILPQ |
| SEQ ID | Amino acid sequence of | SSSSKAPPPSLPSPSRLPGPSDTPILPQLKIAAFNIRT |
| NO: 3 | hyperactive actin resistant | FGRTKMSNATLVSYIVQILSRYDIALVQEVRDSHLTAV |
| | human Dnasel compising | GKLLDNLNQDAPDTYHYVVSEPLGRKSYKERYLFVYRP |
| | one chorionic gonadotropin | DQVSAVDSYYYDDGCEPCGNDTFNREPFIVRFFSRFT |
| | carboxy terminal peptide | EVREFAIVPLHAAPGDAVAEIDALYDVYLDVQEKWGLE |
| | (CTP)-attached to the | DVMLMGDFNAGCSYVRPSQWSSIRLWTSPTFQWLIP |
| | amino terminus and two | DSADTTATPTHCAYDRIVVAGMLLRGAVVPDSALPFNF |
| | chorionic gonadotropin | QAAYGLSDQLAQAISDHYPVEVMLKSSSSKAPPPSLP |
| | carboxy terminal peptides | SPSRLPGPSDTPILPQSSSSKAPPPSLPSPSRLPGPSD |
| | (CTP)-attached to the | TPILPQ |
| | carboxy terminus (CTP3) | |
| SEQ ID | Amino acid sequence of | MRGMKLLGALLALAALLQGAVSLKIAAFNIRTFGRTKM |
| NO: 4 | hyperactive actin resistant | SNATLVSYIVQILSRYDIALVQEVRDSHLTAVGKLLDN |
| | human Dnasel compising | LNQDAPDTYHYVVSEPLGRKSYKERYLFVYRPDQVSAV |
| | three chorionic | DSYYYDDGCEPCGNDTFNREPFIVRFFSRFTEVREFAI |
| | gonadotropin carboxy | VPLHAAPGDAVAEIDALYDVYLDVQEKWGLEDVMLMG |
| | terminal peptides (CTP) | DFNAGCSYVRPSQWSSIRLWTSPTFQWLIPDSADTTA |
| | attached to the carboxy | TPTHCAYDRIVVAGMLLRGAVVPDSALPFNFQAAYGL |
| | terminus (CTP1) with | SDQLAQAISDHYPVEVMLKSSSSKAPPPSLPSPSRLP |
| | leader peptide | GPSDTPILPQSSSSKAPPPSLPSPSRLPGPSDTPILP |
| | | QSSSSKAPPPSLPSPSRLPGPSDTPILPQ |
| SEQ ID | Amino acid sequence of | MRGMKLLGALLALAALLQGAVSLKIAAFNIRTFGRTKM |
| NO: 5 | hyperactive actin resistant | SNATLVSYIVQILSRYDIALVQEVRDSHLTAVGKLLDN |
| | human Dnasel compising | LNQDAPDTYHYVVSEPLGRKSYKERYLFVYRPDQVSAV |
| | two chorionic gonadotropin | DSYYYDDGCEPCGNDTFNREPFIVRFFSRFTEVREFAI |
| | carboxy terminal peptides | VPLHAAPGDAVAEIDALYDVYLDVQEKWGLEDVMLMG |
| | (CTP)-attached to the | DFNAGCSYVRPSQWSSIRLWTSPTFQWLIPDSADTTA |
| | carboxy terminus (CTP2) | TPTHCAYDRIVVAGMLLRGAVVPDSALPFNFQAAYGL |
| | with leader peptide | SDQLAQAISDHYPVEVMLKSSSSKAPPPSLPSPSRLP |
| | | GPSDTPILPQSSSSKAPPPSLPSPSRLPGPSDTPILPQ |
| SEQ ID | Amino acid sequence of | MRGMKLLGALLALAALLQGAVSSSSSKAPPPSLPSPS |
| NO: 6 | hyperactive actin resistant | RLPGPSDTPILPQLKIAAFNIRTFGRTKMSNATLVSYIV |
| | human Dnasel compising | QILSRYDIALVQEVRDSHLTAVGKLLDNLNQDAPDTYH |
| | one chorionic gonadotropin | YVVSEPLGRKSYKERYLFVYRPDQVSAVDSYYYDDGC |
| | carboxy terminal peptide | EPCGNDTFNREPFIVRFFSRFTEVREFAIVPLHAAPGD |
| | (CTP)-attached to the | AVAEIDALYDVYLDVQEKWGLEDVMLMGDFNAGCSY |
| | amino terminus and two | VRPSQWSSIRLWTSPTFQWLIPDSADTTATPTHCAYD |
| | chorionic gonadotropin | RIVVAGMLLRGAVVPDSALPFNFQAAYGLSDQLAQAIS |
| | carboxy terminal peptides | DHYPVEVMLKSSSSKAPPPSLPSPSRLPGPSDTPILPQ |
| | (CTP)-attached to the | SSSSKAPPPSLPSPSRLPGPSDTPILPQ |
| | carboxy terminus (CTP3) | |
| | with leader peptide | |
| SEQ ID | Nucleotide sequence of | AGGCTCAGAGGCACACAGGAGTTTCTGGGCTCACC |
| NO: 7 | ApoEHCR enhancer-hAAT | CTGCCCCCTTCCAACCCCTCAGTTCCCATCCTCCAG |
| | promoter-hDNaseI | CAGCTGTTTGTGTGCTGCCTCTGAAGTCCACACTGA |
| | (hyperactive)- leader | ACAAACTTCAGCCTACTCATGTCCCTAAAATGGGCA |
| | CTP1-WPRE Xinact | AACATTGCAAGCAGCAAACAGCAAACACACAGCCCT |
| | expression cassete | CCCTGCCTGCTGACCTTGGAGCTGGGGCAGAGGTC |
| | | AGAGACCTCTCTGGGCCCATGCCACCTCCAACATC |
| | | CACTCGACCCCTTGGAATTTCGGTGGAGAGGAGCA |
| | | GAGGTTGTCCTGGCGTGGTTTAGGTAGTGTGAGAG |
| | | GGATCTTGCTACCAGTGGAACAGCCACTAAGGATTC |
| | | TGCAGTGAGAGCAGAGGGCCAGCTAAGTGGTACTC |
| | | TCCCAGAGACTGTCTGACTCACGCCACCCCCTCCA |
| | | CCTTGGACACAGGACGCTGTGGTTTCTGAGCCAGG |
| | | TACAATGACTCCTTTCGGTAAGTGCAGTGGAAGCTG |
| | | TACACTGCCCAGGCAAAGCGTCCGGGCAGCGTAGG |
| | | CGGGCGACTCAGATCCCAGCCAGTGGACTTAGCCC |
| | | CTGTTTGCTCCTCCGATAACTGGGGTGACCTTGGTT |
| | | AATATTCACCAGCAGCCTCCCCCGTTGCCCCTCTGG |
| | | ATCCACTGCTTAAATACGGACGAGGACAGGGCCCT |
| | | GTCTCCTCAGCTTCAGGCACCACCACTGACCTGGG |
| | | ACAGTGAATGCCGCCACCATGAGGGGCATGAAGCT |
| | | GCTGGGGGCGCTGCTGGCACTGGCGGCCCTACTG |
| | | CAGGGGGCCGTGTCCCTGAAGATCGCAGCCTTCAA |
| | | CATCAGGACATTTGGGAGGACCAAGATGTCCAATGC |
| | | CACCCTCGTCAGCTACATTGTGCAGATCCTGAGCCG |
| | | CTATGACATCGCCCTGGTCCAGGAGGTCAGAGACA |
| | | GCCACCTGACTGCCGTGGGGAAGCTGCTGGACAAC |
| | | CTCAATCAGGATGCACCAGACACCTATCACTACGTG |
| | | GTCAGTGAGCCACTGGGACGGAAGAGCTATAAGGA |
| | | GCGCTACCTGTTCGTGTACAGGCCTGACCAGGTGT |
| | | CTGCGGTGGACAGCTACTACTACGATGATGGCTGC |
| | | GAGCCCTGCGGGAACGACACCTTCAACCGAGAGCC |
| | | ATTCATTGTCAGGTTCTTCTCCCGGTTCACAGAGGT |
| | | CAGGGAGTTTGCCATTGTTCCCCTGCATGCGGCCC |
| | | CGGGGGACGCAGTAGCCGAGATCGACGCTCTCTAT |
| | | GACGTCTACCTGGATGTCCAAGAGAAATGGGGCTT |
| | | GGAGGACGTCATGTTGATGGGCGACTTCAATGCGG |
| | | GCTGCAGCTATGTGAGACCCTCCCAGTGGTCATCC |
| | | ATCCGCCTGTGGACAAGCCCCACCTTCCAGTGGCT |
| | | GATCCCCGACAGCGCTGACACCACAGCTACACCCA |
| | | CGCACTGTGCCTATGACAGGATCGTGGTTGCAGGG |
| | | ATGCTGCTCCGAGGCGCCGTTGTTCCCGACTCGGC |
| | | TCTTCCCTTTAACTTCCAGGCTGCCTATGGCCTGAG |
| | | TGACCAACTGGCCCAAGCCATCAGTGACCACTATCC |
| | | AGTGGAGGTGATGCTGAAGTCC TCC AGT AGT AAG |
| | | GCC CCA CCT CCC AGT TTG CCC AGT CCT TCC |
| | | AGG TTG CCA GGG CCA AGT GAC ACC CCT ATT |
| | | TTG CCA CAA TCA TCT AGC AGT AAA GCA CCA |
| | | CCA CCT TCT CTT CCT TCT CCC AGC AGA TTG |
| | | CCC GGA CCC TCT GAT ACT CCC ATA TTG CCC |
| | | CAG TCC TCT TCT TCA AAG GCC CCT CCT CCC |
| | | TCA CTT CCA TCT CCT AGC AGG CTT CCC GGA |
| | | CCT TCA GAT ACA CCA ATC CTG CCT CAG |
| | | TGAAGTGGCGGCCGCTCGAGCTAGCGGCCGCTCTA |
| | | GAAGATAATCAACCTCTGGATTACAAAATTTGTGAAA |
| | | GATTGACTGGTATTCTTAACTATGTTGCTCCTTTTAC |
| | | GCTATGTGGATACGCTGCTTTAATGCCTTTGTATCAT |
| | | GCTATTGCTTCCCGTATGGCTTTCATTTTCTCCTCCT |
| | | TGTATAAATCCTGGTTGCTGTCTCTTTATGAGGAGTT |
| | | GTGGCCCGTTGTCAGGCAACGTGGCGTGGTGTGCA |
| | | CTGTGTTTGCTGACGCAACCCCCACTGGTTGGGGC |
| | | ATTGCCACCACCTGTCAGCTCCTTTCCGGGACTTTC |
| | | GCTTTCCCCCTCCCTATTGCCACGGCGGAACTCATC |
| | | GCCGCCTGCCTTGCCCGCTGCTGGACAGGGGCTC |
| | | GGCTGTTGGGCACTGACAATTCCGTGGTGTTGTCG |
| | | GGGAAATCATCGTCCTTTCCTTGGCTGCTCGCCTGT |
| | | GTTGCCACCTGGATTCTGCGCGGGACGTCCTTCTG |
| | | CTACGTCCCTTCGGCCCTCAATCCAGCGGACCTTCC |
| | | TTCCCGCGGCCTGCTGCCGGCTCTGCGGCCTCTTC |
| | | CGCGTCTTCGCCTTCGCCCTCAGACGAGTCGGATC |
| | | TCCCTTTGGGCCGCCTCCCCGCATCGGACTAG |
| SEQ ID | Nucleotide sequence of | AGGCTCAGAGGCACACAGGAGTTTCTGGGCTCACC |
| NO: 8 | ApoEHCR enhancer-hAAT | CTGCCCCCTTCCAACCCCTCAGTTCCCATCCTCCAG |
| | promoter-hDNaseI | CAGCTGTTTGTGTGCTGCCTCTGAAGTCCACACTGA |
| | (hyperactive)- leader | ACAAACTTCAGCCTACTCATGTCCCTAAAATGGGCA |
| | CTP2-WPRE Xinact | AACATTGCAAGCAGCAAACAGCAAACACACAGCCCT |
| | expression cassete | CCCTGCCTGCTGACCTTGGAGCTGGGGCAGAGGTC |
| | | AGAGACCTCTCTGGGCCCATGCCACCTCCAACATC |
| | | CACTCGACCCCTTGGAATTTCGGTGGAGAGGAGCA |
| | | GAGGTTGTCCTGGCGTGGTTTAGGTAGTGTGAGAG |
| | | GGATCTTGCTACCAGTGGAACAGCCACTAAGGATTC |
| | | TGCAGTGAGAGCAGAGGGCCAGCTAAGTGGTACTC |
| | | TCCCAGAGACTGTCTGACTCACGCCACCCCCTCCA |
| | | CCTTGGACACAGGACGCTGTGGTTTCTGAGCCAGG |
| | | TACAATGACTCCTTTCGGTAAGTGCAGTGGAAGCTG |
| | | TACACTGCCCAGGCAAAGCGTCCGGGCAGCGTAGG |
| | | CGGGCGACTCAGATCCCAGCCAGTGGACTTAGCCC |
| | | CTGTTTGCTCCTCCGATAACTGGGGTGACCTTGGTT |
| | | AATATTCACCAGCAGCCTCCCCCGTTGCCCCTCTGG |
| | | ATCCACTGCTTAAATACGGACGAGGACAGGGCCCT |
| | | GTCTCCTCAGCTTCAGGCACCACCACTGACCTGGG |
| | | ACAGTGAATGCCGCCACCATGAGGGGCATGAAGCT |
| | | GCTGGGGGCGCTGCTGGCACTGGCGGCCCTACTG |
| | | CAGGGGGCCGTGTCCCTGAAGATCGCAGCCTTCAA |
| | | CATCAGGACATTTGGGAGGACCAAGATGTCCAATGC |
| | | CACCCTCGTCAGCTACATTGTGCAGATCCTGAGCCG |
| | | CTATGACATCGCCCTGGTCCAGGAGGTCAGAGACA |
| | | GCCACCTGACTGCCGTGGGGAAGCTGCTGGACAAC |
| | | CTCAATCAGGATGCACCAGACACCTATCACTACGTG |
| | | GTCAGTGAGCCACTGGGACGGAAGAGCTATAAGGA |
| | | GCGCTACCTGTTCGTGTACAGGCCTGACCAGGTGT |
| | | CTGCGGTGGACAGCTACTACTACGATGATGGCTGC |
| | | GAGCCCTGCGGGAACGACACCTTCAACCGAGAGCC |
| | | ATTCATTGTCAGGTTCTTCTCCCGGTTCACAGAGGT |
| | | CAGGGAGTTTGCCATTGTTCCCCTGCATGCGGCCC |
| | | CGGGGGACGCAGTAGCCGAGATCGACGCTCTCTAT |
| | | GACGTCTACCTGGATGTCCAAGAGAAATGGGGCTT |
| | | GGAGGACGTCATGTTGATGGGCGACTTCAATGCGG |
| | | GCTGCAGCTATGTGAGACCCTCCCAGTGGTCATCC |
| | | ATCCGCCTGTGGACAAGCCCCACCTTCCAGTGGCT |
| | | GATCCCCGACAGCGCTGACACCACAGCTACACCCA |
| | | CGCACTGTGCCTATGACAGGATCGTGGTTGCAGGG |
| | | ATGCTGCTCCGAGGCGCCGTTGTTCCCGACTCGGC |
| | | TCTTCCCTTTAACTTCCAGGCTGCCTATGGCCTGAG |
| | | TGACCAACTGGCCCAAGCCATCAGTGACCACTATCC |
| | | AGTGGAGGTGATGCTGAAGTCC AGC AGT AGT AAA |
| | | GCT CCA CCC CCT TCC TTG CCA AGT CCC TCT |
| | | CGG CTC CCA GGT CCA AGC GAT ACC CCC ATT |
| | | CTG CCT CAA AGT AGT AGC AGC AAA GCA CCT |
| | | CCT CCT AGT CTT CCA AGT CCA AGT CGA CTG |
| | | CCA GGG CCA AGT GAC ACC CCC ATA TTG CCC |
| | | CAG |
| | | TGAAGTGGCGGCCGCTCGAGCTAGCGGCCGCTCTA |
| | | GAAGATAATCAACCTCTGGATTACAAAATTTGTGAAA |
| | | GATTGACTGGTATTCTTAACTATGTTGCTCCTTTTAC |
| | | GCTATGTGGATACGCTGCTTTAATGCCTTTGTATCAT |
| | | GCTATTGCTTCCCGTATGGCTTTCATTTTCTCCTCCT |
| | | TGTATAAATCCTGGTTGCTGTCTCTTTATGAGGAGTT |
| | | GTGGCCCGTTGTCAGGCAACGTGGCGTGGTGTGCA |
| | | CTGTGTTTGCTGACGCAACCCCCACTGGTTGGGGC |
| | | ATTGCCACCACCTGTCAGCTCCTTTCCGGGACTTTC |
| | | GCTTTCCCCCTCCCTATTGCCACGGCGGAACTCATC |
| | | GCCGCCTGCCTTGCCCGCTGCTGGACAGGGGCTC |
| | | GGCTGTTGGGCACTGACAATTCCGTGGTGTTGTCG |
| | | GGGAAATCATCGTCCTTTCCTTGGCTGCTCGCCTGT |
| | | GTTGCCACCTGGATTCTGCGCGGGACGTCCTTCTG |
| | | CTACGTCCCTTCGGCCCTCAATCCAGCGGACCTTCC |
| | | TTCCCGCGGCCTGCTGCCGGCTCTGCGGCCTCTTC |
| | | CGCGTCTTCGCCTTCGCCCTCAGACGAGTCGGATC |
| | | TCCCTTTGGGCCGCCTCCCCGCATCGGACTAG |
| SEQ ID | Nucleotide sequence of | AGGCTCAGAGGCACACAGGAGTTTCTGGGCTCACC |
| NO: 9 | ApoEHCR enhancer-hAAT | CTGCCCCCTTCCAACCCCTCAGTTCCCATCCTCCAG |
| | promoter-hDNaseI | CAGCTGTTTGTGTGCTGCCTCTGAAGTCCACACTGA |
| | (hyperactive) - leader | ACAAACTTCAGCCTACTCATGTCCCTAAAATGGGCA |
| | CTP3-WPRE Xinact | AACATTGCAAGCAGCAAACAGCAAACACACAGCCCT |
| | expression cassete | CCCTGCCTGCTGACCTTGGAGCTGGGGCAGAGGTC |
| | | AGAGACCTCTCTGGGCCCATGCCACCTCCAACATC |
| | | CACTCGACCCCTTGGAATTTCGGTGGAGAGGAGCA |
| | | GAGGTTGTCCTGGCGTGGTTTAGGTAGTGTGAGAG |
| | | GGATCTTGCTACCAGTGGAACAGCCACTAAGGATTC |
| | | TGCAGTGAGAGCAGAGGGCCAGCTAAGTGGTACTC |
| | | TCCCAGAGACTGTCTGACTCACGCCACCCCCTCCA |
| | | CCTTGGACACAGGACGCTGTGGTTTCTGAGCCAGG |
| | | TACAATGACTCCTTTCGGTAAGTGCAGTGGAAGCTG |
| | | TACACTGCCCAGGCAAAGCGTCCGGGCAGCGTAGG |
| | | CGGGCGACTCAGATCCCAGCCAGTGGACTTAGCCC |
| | | CTGTTTGCTCCTCCGATAACTGGGGTGACCTTGGTT |
| | | AATATTCACCAGCAGCCTCCCCCGTTGCCCCTCTGG |
| | | ATCCACTGCTTAAATACGGACGAGGACAGGGCCCT |
| | | GTCTCCTCAGCTTCAGGCACCACCACTGACCTGGG |
| | | ACAGTGAATGCCGCCACCATGAGGGGCATGAAGCT |
| | | GCTGGGGGCGCTGCTGGCACTGGCGGCCCTACTG |
| | | CAGGGGGCCGTGTCC TCA AGT TCC TCC AAG |
| | | GCA CCC CCA CCT AGT CTT CCT AGC CCC AGT |
| | | CGC CTC CCC GGA CCT TCA GAC ACT CCT ATT |
| | | CTG CCA CAA |
| | | CTGAAGATCGCAGCCTTCAACATCAGGACATTTGGG |
| | | AGGACCAAGATGTCCAATGCCACCCTCGTCAGCTAC |
| | | ATTGTGCAGATCCTGAGCCGCTATGACATCGCCCTG |
| | | GTCCAGGAGGTCAGAGACAGCCACCTGACTGCCGT |
| | | GGGGAAGCTGCTGGACAACCTCAATCAGGATGCAC |
| | | CAGACACCTATCACTACGTGGTCAGTGAGCCACTG |
| | | GGACGGAAGAGCTATAAGGAGCGCTACCTGTTCGT |
| | | GTACAGGCCTGACCAGGTGTCTGCGGTGGACAGCT |
| | | ACTACTACGATGATGGCTGCGAGCCCTGCGGGAAC |
| | | GACACCTTCAACCGAGAGCCATTCATTGTCAGGTTC |
| | | TTCTCCCGGTTCACAGAGGTCAGGGAGTTTGCCATT |
| | | GTTCCCCTGCATGCGGCCCCGGGGGACGCAGTAG |
| | | CCGAGATCGACGCTCTCTATGACGTCTACCTGGATG |
| | | TCCAAGAGAAATGGGGCTTGGAGGACGTCATGTTG |
| | | ATGGGCGACTTCAATGCGGGCTGCAGCTATGTGAG |
| | | ACCCTCCCAGTGGTCATCCATCCGCCTGTGGACAA |
| | | GCCCCACCTTCCAGTGGCTGATCCCCGACAGCGCT |
| | | GACACCACAGCTACACCCACGCACTGTGCCTATGA |
| | | CAGGATCGTGGTTGCAGGGATGCTGCTCCGAGGCG |
| | | CCGTTGTTCCCGACTCGGCTCTTCCCTTTAACTTCC |
| | | AGGCTGCCTATGGCCTGAGTGACCAACTGGCCCAA |
| | | GCCATCAGTGACCACTATCCAGTGGAGGTGATGCT |
| | | GAAGTCA TCT AGT TCC AAG GCT CCT CCC CCT |
| | | TCC TTG CCA TCT CCA TCC CGG TTG CCC GGT |
| | | CCC TCA GAC ACC CCT ATT CTT CCC CAA AGT |
| | | TCC AGT TCT AAA GCT CCT CCC CCC AGT CTT |
| | | CCT TCC CCT AGC AGG CTC CCC GGA CCA TCC |
| | | GAC ACT CCC ATA TTG CCT CAA |
| | | TGAAGTGGCGGCCGCTCGAGCTAGCGGCCGCTCTA |
| | | GAAGATAATCAACCTCTGGATTACAAAATTTGTGAAA |
| | | GATTGACTGGTATTCTTAACTATGTTGCTCCTTTTAC |
| | | GCTATGTGGATACGCTGCTTTAATGCCTTTGTATCAT |
| | | GCTATTGCTTCCCGTATGGCTTTCATTTTCTCCTCCT |
| | | TGTATAAATCCTGGTTGCTGTCTCTTTATGAGGAGTT |
| | | GTGGCCCGTTGTCAGGCAACGTGGCGTGGTGTGCA |
| | | CTGTGTTTGCTGACGCAACCCCCACTGGTTGGGGC |
| | | ATTGCCACCACCTGTCAGCTCCTTTCCGGGACTTTC |
| | | GCTTTCCCCCTCCCTATTGCCACGGCGGAACTCATC |
| | | GCCGCCTGCCTTGCCCGCTGCTGGACAGGGGCTC |
| | | GGCTGTTGGGCACTGACAATTCCGTGGTGTTGTCG |
| | | GGGAAATCATCGTCCTTTCCTTGGCTGCTCGCCTGT |
| | | GTTGCCACCTGGATTCTGCGCGGGACGTCCTTCTG |
| | | CTACGTCCCTTCGGCCCTCAATCCAGCGGACCTTCC |
| | | TTCCCGCGGCCTGCTGCCGGCTCTGCGGCCTCTTC |
| | | CGCGTCTTCGCCTTCGCCCTCAGACGAGTCGGATC |
| | | TCCCTTTGGGCCGCCTCCCCGCATCGGACTAG |
| | | AGGCTCAGAGGCACACAGGAGTTTCTGGGCTCACC |
| | | CTGCCCCCTTCCAACCCCTCAGTTCCCATCCTCCAG |
| | | CAGCTGTTTGTGTGCTGCCTCTGAAGTCCACACTGA |
| | | ACAAACTTCAGCCTACTCATGTCCCTAAAATGGGCA |
| | | AACATTGCAAGCAGCAAACAGCAAACACACAGCCCT |
| | | CCCTGCCTGCTGACCTTGGAGCTGGGGCAGAGGTC |
| | | AGAGACCTCTCTGGGCCCATGCCACCTCCAACATC |
| | | CACTCGACCCCTTGGAATTTCGGTGGAGAGGAGCA |
| | | GAGGTTGTCCTGGCGTGGTTTAGGTAGTGTGAGAG |
| | | GGATCTTGCTACCAGTGGAACAGCCACTAAGGATTC |
| | | TGCAGTGAGAGCAGAGGGCCAGCTAAGTGGTACTC |
| | | TCCCAGAGACTGTCTGACTCACGCCACCCCCTCCA |
| | | CCTTGGACACAGGACGCTGTGGTTTCTGAGCCAGG |
| | | TACAATGACTCCTTTCGGTAAGTGCAGTGGAAGCTG |
| | | TACACTGCCCAGGCAAAGCGTCCGGGCAGCGTAGG |
| | | CGGGCGACTCAGATCCCAGCCAGTGGACTTAGCCC |
| | | CTGTTTGCTCCTCCGATAACTGGGGTGACCTTGGTT |
| | | AATATTCACCAGCAGCCTCCCCCGTTGCCCCTCTGG |
| | | ATCCACTGCTTAAATACGGACGAGGACAGGGCCCT |
| | | GTCTCCTCAGCTTCAGGCACCACCACTGACCTGGG |
| | | ACAGTGAATGCCGCCACCATGAGGGGCATGAAGCT |
| | | GCTGGGGGCGCTGCTGGCACTGGCGGCCCTACTG |
| | | CAGGGGGCCGTGTCC TCA AGT TCC TCC AAG |
| | | GCA CCC CCA CCT AGT CTT CCT AGC CCC AGT |
| | | CGC CTC CCC GGA CCT TCA GAC ACT CCT ATT |
| | | CTG CCA CAA |
| | | CTGAAGATCGCAGCCTTCAACATCAGGACATTTGGG |
| | | AGGACCAAGATGTCCAATGCCACCCTCGTCAGCTAC |
| | | ATTGTGCAGATCCTGAGCCGCTATGACATCGCCCTG |
| | | GTCCAGGAGGTCAGAGACAGCCACCTGACTGCCGT |
| | | GGGGAAGCTGCTGGACAACCTCAATCAGGATGCAC |
| | | CAGACACCTATCACTACGTGGTCAGTGAGCCACTG |
| | | GGACGGAAGAGCTATAAGGAGCGCTACCTGTTCGT |
| | | GTACAGGCCTGACCAGGTGTCTGCGGTGGACAGCT |
| | | ACTACTACGATGATGGCTGCGAGCCCTGCGGGAAC |
| | | GACACCTTCAACCGAGAGCCATTCATTGTCAGGTTC |
| | | TTCTCCCGGTTCACAGAGGTCAGGGAGTTTGCCATT |
| | | GTTCCCCTGCATGCGGCCCCGGGGGACGCAGTAG |
| | | CCGAGATCGACGCTCTCTATGACGTCTACCTGGATG |
| | | TCCAAGAGAAATGGGGCTTGGAGGACGTCATGTTG |
| | | ATGGGCGACTTCAATGCGGGCTGCAGCTATGTGAG |
| | | ACCCTCCCAGTGGTCATCCATCCGCCTGTGGACAA |
| | | GCCCCACCTTCCAGTGGCTGATCCCCGACAGCGCT |
| | | GACACCACAGCTACACCCACGCACTGTGCCTATGA |
| | | CAGGATCGTGGTTGCAGGGATGCTGCTCCGAGGCG |
| | | CCGTTGTTCCCGACTCGGCTCTTCCCTTTAACTTCC |
| | | AGGCTGCCTATGGCCTGAGTGACCAACTGGCCCAA |
| | | GCCATCAGTGACCACTATCCAGTGGAGGTGATGCT |
| | | GAAGTCA TCT AGT TCC AAG GCT CCT CCC CCT |
| | | TCC TTG CCA TCT CCA TCC CGG TTG CCC GGT |
| | | CCC TCA GAC ACC CCT ATT CTT CCC CAA AGT |
| | | TCC AGT TCT AAA GCT CCT CCC CCC AGT CTT |
| | | CCT TCC CCT AGC AGG CTC CCC GGA CCA TCC |
| | | GAC ACT CCC ATA TTG CCT CAA |
| | | TGAAGTGGCGGCCGCTCGAGCTAGCGGCCGCTCTA |
| | | GAAGATAATCAACCTCTGGATTACAAAATTTGTGAAA |
| | | GATTGACTGGTATTCTTAACTATGTTGCTCCTTTTAC |
| | | GCTATGTGGATACGCTGCTTTAATGCCTTTGTATCAT |
| | | GCTATTGCTTCCCGTATGGCTTTCATTTTCTCCTCCT |
| | | TGTATAAATCCTGGTTGCTGTCTCTTTATGAGGAGTT |
| | | GTGGCCCGTTGTCAGGCAACGTGGCGTGGTGTGCA |
| | | CTGTGTTTGCTGACGCAACCCCCACTGGTTGGGGC |
| | | ATTGCCACCACCTGTCAGCTCCTTTCCGGGACTTTC |
| | | GCTTTCCCCCTCCCTATTGCCACGGCGGAACTCATC |
| | | GCCGCCTGCCTTGCCCGCTGCTGGACAGGGGCTC |
| | | GGCTGTTGGGCACTGACAATTCCGTGGTGTTGTCG |
| | | GGGAAATCATCGTCCTTTCCTTGGCTGCTCGCCTGT |
| | | GTTGCCACCTGGATTCTGCGCGGGACGTCCTTCTG |
| | | CTACGTCCCTTCGGCCCTCAATCCAGCGGACCTTCC |
| | | TTCCCGCGGCCTGCTGCCGGCTCTGCGGCCTCTTC |
| | | CGCGTCTTCGCCTTCGCCCTCAGACGAGTCGGATC |
| | | TCCCTTTGGGCCGCCTCCCCGCATCGGACTAG |
| SEQ ID | CTP amino acid sequence | Asp-Pro-Arg-Phe-Gln-Asp-Ser-Ser-Ser- |
| NO: 10 | | Ser-Lys-Ala-Pro-Pro-Pro-Ser-Leu-Pro- |
| | | Ser-Pro-Ser-Arg-Leu-Pro-Gly-Pro- |
| | | Ser-Asp-Thr-Pro-Ile-Leu-Pro-Gln |
| SEQ ID | CTP amino acid sequence | Ser-Ser-Ser-Ser-Lys-Ala-Pro-Pro-Pro- |
| NO: 11 | | Ser-Leu-Pro-Ser-Pro-Ser-Arg-Leu-Pro- |
| | | Gly-Pro-Ser-Asp-Thr-Pro-Ile-Leu-Pro- |
| | | Gln |
| SEQ ID | CTP amino acid sequence | Ser-Ser-Ser-Ser-Lys-Ala-Pro-Pro-Pro- |
| NO: 12 | | Ser-Leu-Pro-Ser-Pro-Ser-Arg |
| SEQ ID | Mature wild-type (WT) | LKIAAFNIQTFGETKMSNATLVSYIVQILSRYDIALVQ |
| NO: 13 | human DNase I (without | EVRDSHLTAVGKLLDNLNQDAPDTYHYVVSEPLGRNSY |
| | secretory signal sequence; | KERYLFVYRPDQVSAVDSYYYDDGCEPCGNDTFNREPA |
| | Genbank Accession No. | IVRFFSRFTEVREFAIVPLHAAPGDAVAEIDALYDVYL |
| | 4AWN_A) | DVQEKWGLEDVMLMGDFNAGCSYVRPSQWSSIRLWTSP |
| | | TFQWLIPDSADTTATPTHCAYDRIVVAGMLLRGAVVPD |
| | | SALPFNFQAAYGLSDQLAQAISDHYPVEVMLK |
| SEQ ID | Mature human DNAse I | LKIAAFNIRTFGRTKMSNATLVSYIVQILSRYDIALV |
| NO: 14 | mutant (without secretory | QEVRDSHLTAVGKLLDNLNQDAPDTYHYVVSEPLGRK |
| | signal sequence); the | SYKERYLFVYRPDQVSAVDSYYYDDGCEPCGNDTFNR |
| | mutated residues as | EPFIVRFFSRFTEVREFAIVPLHAAPGDAVAEIDALY |
| | compared to SEQ ID NO: | DVYLDVQEKWGLEDVMLMGDFNAGCSYVRPSQWSSIR |
| | 4 are in bold and | LWTSPTFQWLIPDSADTTATPTHCAYDRIVVAGMLLR |
| | underlined | GAVVPDSALPFNFQAAYGLSDQLAQAISDHYPVEVMLK |

1. A recombinant adeno-associated virus (rAAV) expression vector comprising:
(i) a capsid protein and
(ii) a nucleic acid segment,
wherein the nucleic acid segment comprises a promoter operably linked to a nucleotide sequence encoding an enzyme with deoxyribonuclease (DNase) activity,
wherein the enzyme comprises at least two chorionic gonadotropin carboxy terminal peptides (CTP).

2. The rAAV expression vector of claim 1, wherein the promoter is a liver-specific promoter.

3. The rAAV expression vector of claim 1, wherein the two chorionic gonadotropin carboxy terminal peptides (CTP) are attached to the amino terminus of the enzyme.

4. The rAAV expression vector of claim 1, wherein the two chorionic gonadotropin carboxy terminal peptides (CTP) are attached to the carboxy terminus of the enzyme.

5. The rAAV expression vector of claim 1, wherein the capsid protein mediates efficient and/or preferential targeting of the vector to the liver when administered in vivo.

6. The rAAV expression vector of claim 2, wherein the capsid protein mediates efficient and/or preferential targeting of the vector to the liver when administered in vivo.

7. The rAAV expression vector of claim 1, wherein the capsid protein is LK03. The rAAV expression vector of claim 1, wherein the enzyme with Dnase activity is selected from the group consisting of Dnase I, Dnase X, Dnase γ, Dnase1L1, Dnase1L2, Dnase 1L3, Dnase II, Dnase liα, Dnase liβ, Caspase-activated Dnase (CAD), DNA fragmentation factor B, L Dnase II, Endonuclease G (ENDOG), Granzyme B and mutants or derivatives thereof.

8. The rAAV expression vector of claim 1, wherein the enzyme with DNase activity is human DNase I or a mutant or derivative thereof.

9. The rAAV expression vector of claim 1, wherein the enzyme with DNase activity is comprised of an amino acid sequence having at least 95% sequence identity to SEQ ID NO: 1, SEQ ID NO: 2 or SEQ ID NO: 3.

10. A method for treating a disease or condition in a subject in need thereof, the method comprising administering to the subject a therapeutically effective amount of the rAAV expression vector of claim 1,
wherein the disease or condition is accompanied by intravascular or extravascular accumulation of extracellular DNA.

11. The method of claim 11, where the disease or condition is one or more of cancer (e.g., carcinomas, sarcomas, lymphomas, melanoma), an autoimmune disease, development of somatic mosaicism, side effects associated with chemotherapy or radiation therapy, a neurodegenerative disease, an infection, diabetes, a cardiovascular disease (e.g., atherosclerosis, stroke, angina, ischemia), kidney damage, delayed-type hypersensitivity reactions (e.g., graft-versus-host disease, reduction of fertility, age-specific sperm motility impairment and aging.

12. The method of claim 11, wherein the therapeutically effective dose of the rAAV is below 1.0×10¹⁴ GC/kg.

13. The method of claim 11, wherein the therapeutically effective dose of the rAAV is below 1.0×10¹³ GC/kg.

14. The method of claim 11, wherein administering to the subject of the rAAV vector results in liver expression of DNase enzyme having at least one of the chorionic gonadotropin carboxy terminal peptides (CTP) glycosylated via glycosylation.

15. The method of claim 16, wherein the glycosylation comprises O-linked glycans.

16. The method of claim 16, wherein at least one molecule of sialic acid is added to a CTP-modified DNase amino acid sequence following glycosylation.

17. The method of claim 16, wherein about 1 to 10 sialic acid molecules are added per each CTP-modified DNase amino acid sequence.

18. The method of claim 16, wherein about 1 to 120 sialic acid molecules are added in total per each CTP-modified DNase amino acid sequence.

19. The method of claim 16, wherein the CTP amino acid sequence is at least 70% homologous to SEQ ID NO: 10.

20. The method of claim 16, wherein the CTP amino acid sequence is at least 70% homologous to SEQ ID NO: 11.

21.-58. (canceled)


SEQUENCE LISTING (14 sequences)

SEQ ID NO: 1 - protein, 344 aa, synthetic construct
  REGION 1..344: note = Amino acid sequence of hyperactive actin resistant human DnaseI compising three chorionic gonadotropin carboxy terminal peptides (CTP) attached to the carboxy terminus (CTP1)
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 2 - protein, 316 aa, synthetic construct
  REGION 1..316: note = Amino acid sequence of hyperactive actin resistant human DnaseI compising two chorionic gonadotropin carboxy terminal peptides (CTP) attached to the carboxy terminus (CTP2)
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQ

SEQ ID NO: 3 - protein, 344 aa, synthetic construct
  REGION 1..344: note = Amino acid sequence of hyperactive actin resistant human DnaseI compising one chorionic gonadotropin carboxy terminal peptide (CTP) attached to the amino terminus and two chorionic gonadotropin carboxy...
SSSSKAPPPSLPSPSRLPGPSDTPILPQLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQ

SEQ ID NO: 4 - protein, 366 aa, synthetic construct
  REGION 1..366: note = Amino acid sequence of hyperactive actin resistant human DnaseI compising three chorionic gonadotropin carboxy terminal peptides (CTP) attached to the carboxy terminus (CTP1) with leader peptide
MRGMKLLGALLALAALLQGAVSLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQSSSSKAPPPSLPSPSRLPGPSDTPILPQ

SEQ ID NO: 5 - protein, 338 aa, synthetic construct
  REGION 1..338: note = Amino acid sequence of hyperactive actin resistant human DnaseI compising two chorionic gonadotropin carboxy terminal peptides (CTP) attached to the carboxy terminus (CTP2) with leader peptide
MRGMKLLGALLALAALLQGAVSLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQ

SEQ ID NO: 6 - protein, 366 aa, synthetic construct
  REGION 1..366: note = Amino acid sequence of hyperactive actin resistant human DnaseI compising one chorionic gonadotropin carboxy terminal peptide (CTP) attached to the amino terminus and two ...
MRGMKLLGALLALAALLQGAVSSSSSKAPPPSLPSPSRLPGPSDTPILPQLKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKSSSSKAPPPSLPSPSRLPGPSDTPILPQSSSSKAPPPSLPSPSRLPGPSDTPILPQ

SEQ ID NO: 7 - DNA, 2464 bp, synthetic construct
  misc_feature 1..2464: note = Nucleotide sequence of ApoEHCR enhancer-hAAT promoter-hDNaseI (hyperactive)- leader CTP1-WPRE Xinact expression cassete
  source 1..2464: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtcctccagtagtaaggccccacctcccagtttgcccagtccttccaggttgccagggccaagtgacacccctattttgccacaatcatctagcagtaaagcaccaccaccttctcttccttctcccagcagattgcccggaccctctgatactcccatattgccccagtcctcttcttcaaaggcccctcctccctcacttccatctcctagcaggcttcccggaccttcagatacaccaatcctgcctcagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 8 - DNA, 2380 bp, synthetic construct
  misc_feature 1..2380: note = Nucleotide sequence of ApoEHCR enhancer-hAAT promoter-hDNaseI (hyperactive)- leader CTP2-WPRE Xinact expression cassete
  source 1..2380: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtccctgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtccagcagtagtaaagctccacccccttccttgccaagtccctctcggctcccaggtccaagcgatacccccattctgcctcaaagtagtagcagcaaagcacctcctcctagtcttccaagtccaagtcgactgccagggccaagtgacacccccatattgccccagtgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 9 - DNA, 4928 bp, synthetic construct
  misc_feature 1..4928: note = Nucleotide sequence of ApoEHCR enhancer-hAAT promoter-hDNaseI (hyperactive)- leader CTP3-WPRE Xinact expression cassete
  source 1..4928: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtcctcaagttcctccaaggcacccccacctagtcttcctagccccagtcgcctccccggaccttcagacactcctattctgccacaactgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtcatctagttccaaggctcctcccccttccttgccatctccatcccggttgcccggtccctcagacacccctattcttccccaaagttccagttctaaagctcctccccccagtcttccttcccctagcaggctccccggaccatccgacactcccatattgcctcaatgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccgccaccatgaggggcatgaagctgctgggggcgctgctggcactggcggccctactgcagggggccgtgtcctcaagttcctccaaggcacccccacctagtcttcctagccccagtcgcctccccggaccttcagacactcctattctgccacaactgaagatcgcagccttcaacatcaggacatttgggaggaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcgggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtcatctagttccaaggctcctcccccttccttgccatctccatcccggttgcccggtccctcagacacccctattcttccccaaagttccagttctaaagctcctccccccagtcttccttcccctagcaggctccccggaccatccgacactcccatattgcctcaatgaagtggcggccgctcgagctagcggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactag

SEQ ID NO: 10 - protein, 33 aa, synthetic construct
  REGION 1..33: note = CTP amino acid sequence
NPRFQNSSSSKAPPPSLPSPSRLPGPSNTILPQ

SEQ ID NO: 11 - protein, 28 aa, synthetic construct
  REGION 1..28: note = CTP amino acid sequence
SSSSKAPPPSLPSPSRLPGPSNTPILPQ

SEQ ID NO: 12 - protein, 16 aa, synthetic construct
  REGION 1..16: note = CTP amino acid sequence
SSSSKAPPPSLPSPSR

SEQ ID NO: 13 - protein, 260 aa, synthetic construct
  REGION 1..260: note = Mature wild-type (WT) human DNase I (without secretory signal sequence; Genbank Accession No.4AWN_A)
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 14 - protein, 260 aa, synthetic construct
  REGION 1..260: note = Mature human DNAse I mutant (without secretory signal sequence); the mutated residues as compared to SEQ ID NO: 4 are in bold and underlined
LKIAAFNIRTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK